(19) United States
(12) Patent Application Publication
(10) Pub. No.: US 2023/0194410 A1
(43) Pub. Date: Jun. 22, 2023

(54) METHOD FOR DETECTING ANTIGEN-SPECIFIC ACTIVATED T CELL

(71) Applicant: SYSMEX CORPORATION, Kobe-shi (JP)

(72) Inventors: Toyoko KATAYAMA, Kobe-shi (JP); Hitoshi UGA, Kobe-shi (JP); Akio OHTA, Kobe-shi (JP); Masaki TAJIMA, Kobe-shi (JP); Naoko IKUTA, Kobe-shi (JP)

(73) Assignee: SYSMEX CORPORATION, Kobe-shi (JP)

(21) Appl. No.: 18/079,556

(22) Filed: Dec. 12, 2022

(30) Foreign Application Priority Data
Dec. 17, 2021 (JP) 2021-205577

(51) Int. Cl. G01N 15/14; G01N 33/49; G01N 33/58; G06T 7/00

(52) U.S. Cl. CPC G01N 15/1475; G01N 15/147; G01N 15/1434; G01N 33/582; G01N 33/4915; G06T 7/0012; G01N 2015/1006

(57) ABSTRACT

Disclosed is a method for detecting an antigen-specific activated T cell comprising: acquiring first information on a particle size and second information on a T cell activation marker for a first measurement sample prepared by mixing in vitro a first specimen separated from a biological sample containing a T cell and an antigen-presenting cell and an antigen reagent containing a predetermined antigen, by measuring the first measurement sample with a flow cytometer; acquiring the first information and the second information for a second measurement sample prepared from a second specimen separated from the biological sample and not containing the antigen reagent, by measuring the second measurement sample with the flow cytometer; detecting a target particle in the first measurement sample based on the first information and the second information on the first measurement sample, and detecting a background particle in the second measurement sample based on the first information and the second information on the second measurement sample; and detecting a cell complex in which the T cell and the antigen-presenting cell adhere to each other in the first measurement sample, the cell complex including a T cell activated by the predetermined antigen, based on a detection result of the target particle and a detection result of the background particle.

### CROSS REFERENCE TO RELATED APPLICATIONS

This application claims priority from prior Japanese Patent Application No. 2021-205577, filed on Dec. 17, 2021, entitled “Method for detecting antigen-specific activated T cell, method for assisting determination of specific immune response to antigen, method for assisting determination of immune tolerance of subject, reagent kit, analysis device, and computer program”, the entire contents of which are incorporated herein by reference.

### FIELD OF THE INVENTION

The present invention relates to a method for detecting an antigen-specific activated T cell.

### BACKGROUND

In recent years, detection of an immune synapse has attracted attention as one of indices of stimulation response of an immune cell. An immune synapse is a structure formed on an immune cell at a contact surface when the immune cell comes into contact with a target cell or a foreign substance having an immunostimulator. For example, U.S. Pat. Application Publication No. 2018/0292385 describes a method for measuring an immunostimulatory response of an immune cell by contacting the immune cell with a foreign substance having an immunostimulator to stimulate the immune cell, eliminating the contact surface, and then detecting an immune synapse remaining on the contact surface.

An immune synapse is also formed between a T cell and an antigen-presenting cell. For example, an antigen-presenting cell presenting an antigen by an MHC (Major histocompatibility complex) molecule adheres to a T cell, and the T cell recognizes the antigen by a TCR (T cell receptor) on the cell surface. At this time, an immune synapse composed of a TCR and an MHC molecule containing an antigen is formed between the T cell and the antigen-presenting cell. When the antigen is an exogenous antigen such as a protein derived from a pathogen, formation of an immune synapse activates the T cell.

However, immune synapse formation between a T cell and an antigen-presenting cell does not necessarily activate the T cell. For example, when the antigen is an autoantigen, an allergen or the like, the T cell is not activated in a healthy subject. This is because a healthy subject is in a state of immune tolerance to a self-antigen and an allergen. Also, some cancer patients may have immune tolerance to a cancer cell. Normally, when a T cell recognizes a cancer antigen on a cancer cell by a TCR, the T cell is activated to attack the cancer cell. However, when PD-1 (Programmed cell death-1) is expressed in a T cell, PD-L1 (Programmed cell death-ligand 1) on a cancer cell binds to PD-1, whereby activation of the T cell is suppressed and immune tolerance to the cancer cell is achieved.

### SUMMARY OF THE INVENTION

The scope of the present invention is defined solely by the appended claims, and is not affected to any degree by the statements within this summary.

As described above, immune synapse formation between a T cell and an antigen-presenting cell does not necessarily mean that the T cell is activated. Therefore, an object of the present inventors is to provide a novel means capable of selectively detecting a cell complex containing a T cell activated by an antigen among cell complexes in which a T cell and an antigen-presenting cell adhere to each other.

The present invention provides a method for detecting an antigen-specific activated T cell including: acquiring first information on a particle size and second information on a T cell activation marker for a first measurement sample prepared by mixing in vitro a first specimen separated from a biological sample containing a T cell and an antigen-presenting cell and an antigen reagent containing a predetermined antigen, by measuring the first measurement sample with a flow cytometer; acquiring first information on a particle size and second information on a T cell activation marker for a second measurement sample prepared from a second specimen separated from the biological sample and not containing the antigen reagent, by measuring the second measurement sample with the flow cytometer; detecting a target particle in the first measurement sample based on the first information and the second information on the first measurement sample, and detecting a background particle in the second measurement sample based on the first information and the second information on the second measurement sample; and detecting a cell complex in which the T cell and the antigen-presenting cell adhere to each other in the first measurement sample, the cell complex including a T cell activated by the predetermined antigen, based on a detection result of the target particle and a detection result of the background particle.

### BRIEF DESCRIPTION OF THE DRAWINGS

FIG. 1A is a view showing an example of a reagent kit of the present embodiment;

FIG. 1B is a view showing an example of a reagent kit of the present embodiment;

FIG. 2 is a view showing a configuration of a cell analyzer;

FIG. 3 is a diagram showing a configuration of a measuring device;

FIG. 4 is a diagram showing a configuration of an imaging unit;

FIG. 5 is a diagram showing a configuration of an analysis device of the present embodiment;

FIGS. 6A and 6B are flowcharts showing measurement processing and analysis processing of a measurement sample;

FIG. 7A shows schematic views of a bright field image, a fluorescence image 1, a fluorescence image 2 and a merge image thereof of a cell complex of an activated T cell and an antigen-presenting cell, which are imaged by an imaging flow cytometer (IFC), and in the figure, MTDR refers to MitoTracker (registered trademark) Deep Red, and FITC refers to fluorescein isothiocyanate;

FIG. 7B shows schematic views of a bright field image, a fluorescence image 1, a fluorescence image 2, and a merge image thereof of a cell complex of a non-activated T cell and an antigen-presenting cell, which are imaged by IFC;

FIG. 8 is a flowchart showing analysis processing by a cell analyzer of Embodiment 1;

FIG. 9 is a schematic diagram of a scattergram to be created in the analysis processing by the cell analyzer of Embodiment 1;

FIG. 10A is a flowchart showing analysis processing of the cell analyzer of Embodiment 1;

FIG. 10B is a flowchart showing analysis processing of the cell analyzer of Embodiment 1;

FIG. 11 is a diagram showing a configuration of a detection unit as a modification of the imaging unit;

FIG. 12 is a flowchart showing analysis processing by a cell analyzer of Embodiment 2;

FIG. 13 is a schematic diagram of a scattergram to be created in the analysis processing by the cell analyzer of Embodiment 2;

FIG. 14 shows scattergrams when a measurement sample to which ovalbumin (OVA) peptide as an antigen has been added to a mixture of a T cell activated by OVA and a B cell and a measurement sample to which OVA peptide has not been added are measured by IFC, and the scattergrams were created based on fluorescence intensity of fluorescently-stained mitochondria and area of the particle;

FIG. 15 shows an image of a cell complex of a T cell containing activated mitochondria and a B cell and a schematic diagram thereof;

FIG. 16 is a graph showing frequency of particles having an area value corresponding to two to three cells in a measurement sample;

FIG. 17 is a graph showing frequency of particles having an area value corresponding to two to three cells in a measurement sample to which an anti-TCR antibody has been added;

FIG. 18 shows scattergrams when a measurement sample to which a viral antigen has been added to a peripheral blood mononuclear cell (PBMC) reactive to the viral antigen and a sample to which a viral antigen has not been added are measured by IFC, and the scattergrams were created based on fluorescence intensity of fluorescently-stained phosphorylated ERK (pERK) and area of the particle;

FIG. 19 shows an image of a cell complex of a T cell containing pERK and a B cell and a schematic diagram thereof;

FIG. 20 shows images of cell complexes in measurement samples in which PBMCs having reactivity different from each other and various exogenous antigens are mixed;

FIG. 21A is a graph showing the number of cell complexes containing an activated T cell per 1 × 10⁴ cells when a measurement sample in which a viral antigen has been mixed with each of PBMC highly reactive with a viral antigen and PBMC low reactive with a viral antigen is measured by IFC;

FIG. 21B is a graph showing the number of spots per 4 × 10⁵ cells when a measurement sample in which a viral antigen has been added to each of PBMC highly reactive with a viral antigen and PBMC low reactive with a viral antigen is measured by ELISpot method;

FIG. 22A is a graph showing the number of cell complexes containing an activated T cell per 1 × 10⁴ cells when a measurement sample in which a viral antigen has been mixed with each of PBMC highly reactive with a Mycobacterium tuberculosis antigen and PBMC low reactive with a viral antigen is measured by IFC;

FIG. 22B is a graph showing the number of spots per 4 × 10⁵ cells when a measurement sample in which a viral antigen has been added to each of PBMC highly reactive with a viral antigen and PBMC low reactive with a Mycobacterium tuberculosis antigen is measured by ELISpot method;

FIG. 23A is a graph showing the number of cell complexes containing an activated T cell per 1 × 10⁴ cells when a measurement sample in which a viral antigen has been mixed with each of PBMC highly reactive with a mite antigen and PBMC low reactive with a viral antigen is measured by IFC;

FIG. 23B is a graph showing the number of spots per 4 × 10⁵ cells when a measurement sample in which a viral antigen has been added to each of PBMC highly reactive with a viral antigen and PBMC low reactive with a mite antigen is measured by ELISpot method;

FIG. 24A is a graph showing the number of cell complexes containing an activated T cell per 1 × 10⁴ cells when a measurement sample in which a type I diabetes antigen or a viral antigen has been mixed with PBMC derived from a type I diabetic patient is measured by IFC;

FIG. 24B is a graph showing the number of spots per 3 × 10⁵ cells when a measurement sample in which a type I diabetes antigen or a viral antigen has been added to PBMC derived from a type I diabetic patient is measured by ELISpot method;

FIG. 25A is a graph showing the number of cell complexes containing an activated T cell per 1 × 10⁴ cells when a measurement sample to which an anti-MHC-II antibody has been added and a measurement sample to which an anti-MHC-II antibody has not been added is measured by IFC;

FIG. 25B is a graph showing the number of spots per 1.5 × 10⁵ cells when a measurement sample to which an anti-MHC-11 antibody has been added is measured by ELISpot method;

FIG. 26A is a graph showing a relationship between the fluorescence intensity derived from labeled mitochondria and the standardized frequency of the cell complex when a measurement sample containing a T cell activated by OVA and a B cell expressing PD-L1 is measured by IFC, and a schematic diagram on the right of the graph shows cell complexes considered to be contained in each measurement sample;

FIG. 26B is a graph showing a result of counting the number of cell complexes having a fluorescence intensity value greater than or equal to a predetermined fluorescence intensity value indicated by a dotted line in the graph of FIG. 26A and calculating a ratio thereof;

FIG. 27 shows scattergrams when a measurement sample to which a viral antigen has been added to PBMC reactive to the viral antigen and a measurement sample to which a viral antigen has not been added are measured by IFC;

FIG. 28 is a graph showing a signal/noise (S/N) ratio calculated from a detection result of a cell complex or an immune synapse in a measurement sample to which a viral antigen has been added to PBMC reactive to the viral antigen and a measurement sample to which a viral antigen has not been added;

FIG. 29 shows scattergrams when a measurement sample to which nivolumab (anti-PD-1 antibody) has been added and a measurement sample to which nivolumab has not been added are measured by IFC, and the scattergrams were created based on fluorescence intensity of fluorescently-stained pERK and area of the particle;

FIG. 30A is a graph showing the number of cell complexes containing an activated T cell per 1 × 10⁴ cells when a measurement sample to which nivolumab has been added and a measurement sample to which nivolumab has not been added is measured by IFC;

FIG. 30B is a graph showing the number of cell complexes per 1 × 10⁴ cells when a measurement sample to which nivolumab has been added and a measurement sample to which nivolumab has not been added is measured by IFC;

FIG. 31 shows scattergrams when a measurement sample to which nivolumab has been added and a measurement sample to which nivolumab has not been added are measured by IFC, and the scattergrams were created based on fluorescence intensity of fluorescently-stained F-actin and area of the particle;

FIG. 32A is a graph showing the number of cell complexes forming an immune synapse per 1 × 10⁴ cells when a measurement sample to which nivolumab has been added and a measurement sample to which nivolumab has not been added is measured by IFC;

FIG. 32B is a graph showing the number of cell complexes per 1 × 10⁴ cells when a measurement sample to which nivolumab has been added and a measurement sample to which nivolumab has not been added is measured by IFC;

FIG. 33 is a graph showing a proportion of PD-1-positive cells in CD4-positive T cells in PBMC stimulated with CD3/CD28 beads;

FIG. 34 shows scattergrams when a measurement sample to which nivolumab has been added and a measurement sample to which nivolumab has not been added are measured by IFC, and the scattergrams were created based on fluorescence intensity of fluorescently-stained mitochondria and area of the particle;

FIG. 35 is a graph showing the number of cell complexes containing an activated T cell per 1 × 10⁴ cells when a measurement sample to which nivolumab has been added and a measurement sample to which nivolumab has not been added is measured by IFC;

FIG. 36A is a graph showing the number of cell complexes containing an activated T cell per 1 × 10⁴ cells detected by algorithm analysis of measurement data;

FIG. 36B is a graph showing the number of cell complexes containing an activated T cell per 1 × 10⁴ cells detected by visual confirmation of an image;

FIG. 37A is a graph showing the number of cell complexes containing an activated T cell per 1 × 10⁴ cells detected by algorithm analysis of measurement data;

FIG. 37B is a graph showing the number of cell complexes containing an activated T cell per 1 × 10⁴ cells detected by visual confirmation of an image; and

FIG. 38 is a graph showing a correlation between a detection result by algorithm analysis of measurement data and a detection result by visual confirmation of an image with respect to the number of cell complexes containing an activated T cell per 1 × 10⁴ cells.

### DETAILED DESCRIPTION OF THE PREFERRED EMBODIMENTS

In the method for detecting an antigen-specific activated T cell of the present embodiment (hereinafter, referred to as “the detection method of the present embodiment”), a first measurement sample prepared by mixing in vitro a first specimen separated from a biological sample containing a T cell and an antigen-presenting cell (first aliquot) and an antigen reagent containing a predetermined antigen is used. In addition, a second measurement sample prepared from a second specimen separated from the same biological sample (second aliquot) and not containing the antigen reagent is used. Specifically, the first measurement sample and the second measurement sample are each measured by a flow cytometer, and first information on a particle size (hereinafter, simply referred to as “first information”) and second information on the T cell activation marker (hereinafter, simply referred to as “second information”) are acquired for each measurement sample.

The biological sample is not particularly limited as long as it can be collected from a subject and can contain a T cell and an antigen-presenting cell. Examples of such a biological sample include samples such as blood, lymph, tissue fluid, joint fluid, bone marrow fluid and saliva, and various tissues. When blood is used as the biological sample, an anticoagulant such as heparin, citrate or ethylenediaminetetraacetate may be added to the blood as necessary. A fraction containing a T cell and an antigen-presenting cell prepared from blood by a conventional method may be used as the biological sample. Example of such a fraction prepared from blood include PBMC, buffy coat, and the like. In the present specification, blood and a fraction prepared from blood are also referred to as “blood specimen”.

The biological sample or specimen may be stored in a suitable solution such that the T cell and the antigen-presenting cell are maintained in a viable state. Examples of such a liquid include a medium generally used for culturing an immune cell in vitro. Such a medium is known, and examples thereof include MEM, DMEM, RPMI-1640, CTL-Test medium (Cellular Technology Limited), and the like. If necessary, the medium may be mixed with an additive such as fetal bovine serum (FBS) or L-glutamine. In a preferred embodiment, the biological sample or specimen is stored in a medium under conditions under which an immune cell can be cultured. Such conditions are known per se, and examples thereof include conditions at 37° C. in a 5% CO₂ atmosphere.

The subject may be a healthy person, a person suffering from a disease, a person infected with a pathogen, or a person suspected of suffering from a disease or infection with a pathogen.

The antigen-presenting cell is not particularly limited as long as it is a cell that is present in the biological sample and can present a predetermined antigen described later. The antigen-presenting cell may be a single cell or a subpopulation (subset) substantially composed of a plurality of homogeneous cells. The antigen-presenting cell is preferably a cell having an MHC class II molecule (an HLA class II molecule in human), and more preferably a professional antigen-presenting cell. Examples of the antigen-presenting cell include a B cell, a dendritic cell, a macrophage (or a monocyte), and the like. The antigen-presenting cell may be one type or two or more types.

Examples of the T cell include effector T cells such as helper T cells (also referred to as CD4⁺ T cells), regulatory T cells (also referred to as T_{reg} cells) and cytotoxic T cells (also referred to as killer T cells or CD8⁺ T cells), naive T cells, genetically-modified T cells, and the like. The effector T cell may be a T cell activated either in vivo or in vitro. The T cell may be a single cell or a subset substantially composed of a plurality of homogeneous T cells. The T cell may be one type or two or more types.

The predetermined antigen is not particularly limited, and may be any antigen for which the state of immune tolerance in a subject is desired to be examined. The predetermined antigen may be any substance as long as it has antigenicity, and examples thereof include proteins, peptides, amino acids, nucleic acids, saccharides, lipids, chemical substances, metals, metal ions, complexes thereof, and the like. In one embodiment, examples of the predetermined antigen include proteins contained in a pathogen or fragments of the protein, allergens, drugs, autoantigens, cancer antigens, and the like. The predetermined antigen may be one type or two or more types.

The pathogen is not particularly limited as long as it is an organism that can cause a disease, and examples thereof include bacteria, fungi, viruses, chlamydia, mycoplasma, spirochetes, rickettsia, protozoa, parasites, and the like. Examples of the protein contained in the pathogen include proteins constituting a part of the pathogen, proteins contained in an extract of the pathogen, and proteins contained in a secretion of the pathogen. The allergen is not particularly limited as long as it is a substance that can cause allergy, and examples thereof include proteins derived from plants, animals, foods, house dust, mold, and the like, and fragments thereof. The drug is not particularly limited, and examples thereof include pharmaceutical compounds and pharmaceutical compositions known to have caused an abnormality of the immune system such as drug eruption in the past. The autoantigen is not particularly limited as long as it is an antigen against an autoantibody that can cause an autoimmune disease. Examples of the autoantigen include IA-2 (Insulinoma associated antigen-2), glutamic acid decarboxylase, insulin, and the like. The cancer antigen is not particularly limited as long as it is a protein specifically expressed in a cancer cell and a fragment thereof. Examples of the cancer antigen include tumor-associated antigens such as differentiation antigens, embryonic proteins, glycoproteins, glycolipids and sugar chain antigens, neoantigens, cancer testis antigens, viral antigens, and the like.

The antigen reagent containing a predetermined antigen (hereinafter, also referred to as “antigen reagent”) is a reagent prepared so that the predetermined antigen can come into contact with a T cell and an antigen-presenting cell when mixed with the first specimen in vitro. For example, the antigen reagent may be a liquid reagent in which a predetermined antigen is dissolved or suspended in an appropriate solvent. Such a solvent is not particularly limited, but is preferably an aqueous solvent. Examples of the aqueous solvent include water, physiological saline, phosphate buffered saline (PBS), Good’s buffer, and the like. Also, the medium may be used as a solvent. The concentration of the antigen in the antigen reagent that is a liquid reagent can be appropriately determined according to the type of antigen. For example, when the predetermined antigen is a protein or a fragment thereof, the concentration of the antigen in the antigen reagent can be determined from the range of 0.1 µg/mL or more and 500 µg/mL or less.

The first measurement sample can be prepared by mixing the first specimen that is a part of the biological sample collected from a subject and an antigen reagent in vitro. In vitro mixing can be performed, for example, by placing a liquid containing a predetermined antigen and the first specimen stored in a medium in an appropriate container, and stirring them by inversion mixing, pipetting, a shaker, or the like. The container is not particularly limited as long as it can be used for culturing or storing a cell. The container may be, for example, a container for cell culture such as a dish, a multi-well plate, a flask or a bottle, or a container used for an experiment using cells such as a tube or a syringe.

As described above, the second measurement sample is prepared from the second specimen that is a part of the biological sample collected from a subject, and does not contain an antigen reagent. That is, in the preparation of the second measurement sample, the antigen reagent is not added to the second specimen. For example, the second measurement sample can be prepared by mixing the second specimen and an appropriate solvent in vitro. The solvent and mixing in vitro are as described above. When the antigen reagent used for preparing the first measurement sample is a liquid reagent, the solvent used for the reagent may be used for preparing the second measurement sample. In the present embodiment, the second measurement sample is preferably prepared under the same conditions as the first measurement sample except that the antigen reagent is not used. The first specimen and the second specimen are specimens separated from the same biological sample.

In the first measurement sample, the following immune reaction may occur. First, an antigen-presenting cell comes into contact with a predetermined antigen and incorporates the antigen into the cell. Then, the antigen-presenting cell presents all or a part of a predetermined antigen. When a T cell recognizes an antigen presented by an antigen-presenting cell via TCR, the T cell and the antigen-presenting cell adhere to each other to form a cell complex. At this time, antigen-specific activity is generated in the T cell in the cell complex. The antigen-specific activity generated in the T cell is reflected in a T cell activation marker. In the detection method of the present embodiment, the cell complex in which the T cell and the antigen-presenting cell adhere to each other is selectively detected based on the particle size measured by a flow cytometer. Then, based on the T cell activation marker measured by the flow cytometer, a cell complex containing an activated T cell is selectively detected from the cell complex in which the T cell and the antigen-presenting cell adhere to each other.

The first measurement sample may contain a particle with approximately the same size as the cell complex in which the T cell and the antigen-presenting cell adhere to each other. Examples thereof include aggregates of cells formed by non-specific adhesion, lipid particles, aggregates of impurities, and the like. In addition, the first measurement sample may also contain a cell complex containing a T cell that specifically reacts with an antigen other than the predetermined antigen (for example, a substance in a medium). Furthermore, among these particles, there may be a particle that nonspecifically reacts with a labeling substance used for labeling a T cell activity marker described later. Thus, these particles become a background for flow cytometer measurement and prevent selective detection of a cell complex containing an activated T cell. In order to selectively detect a cell complex containing an activated T cell in the first measurement sample, it is necessary to remove the background. Therefore, in the detection method of the present embodiment, the second measurement sample is used. Since the second measurement sample does not contain an antigen reagent, it is considered that a cell complex containing a T cell activated by a predetermined antigen in the antigen reagent is not present in the second measurement sample. Therefore, the measurement result of the second measurement sample by the flow cytometer reflects the background.

In order to promote the immune reaction in the first measurement sample, it is preferable that the first specimen and the antigen reagent are mixed in vitro, and then the first measurement sample is incubated under conditions under which an immune cell can be cultured. The conditions under which an immune cell can be cultured are as described above. The incubation time is not particularly limited, but the upper limit is, for example, 6 hours, preferably 5 hours, and more preferably 4 hours. The lower limit of the incubation time is, for example, 10 minutes, preferably 1 hour, and more preferably 2 hours. The incubation time refers to, for example, a time from when the first specimen and the antigen reagent are mixed in vitro until the cell in the first measurement sample is fixed. In order to make the measurement conditions the same as those of the first measurement sample, it is preferable to incubate the second measurement sample in the same manner.

It is preferable to fix the cell in each measurement sample before measuring the first measurement sample and the second measurement sample with the flow cytometer. By performing a fixation treatment of the cell, for example, it is possible to maintain a state change in the T cell due to antigen-specific activity. The fixation of the cell can be performed by adding a fixing agent to the measurement sample. The fixing agent may be a reagent usually used for immunostaining of cell or tissue, and examples thereof include paraformaldehyde (PFA), lower alcohols, and the like. A commercially available cell fixing solution may also be used.

Each of the first measurement sample and the second measurement sample is measured by the flow cytometer to acquire first information and second information for each measurement sample. As used herein, the term “flow cytometer” includes an imaging flow cytometer. Hereinafter, when a conventional flow cytometer without an imaging unit is particularly referred to, it is described as “FCM”, and when an imaging flow cytometer is particularly referred to, it is described as “IFC”.

The FCM is a device that irradiates an individual particle in a liquid flowing in a flow cell with light and detects scattered light and/or fluorescence emitted from each particle as an optical signal. When the particle is a cell, the size of each cell, distribution of the amount of molecules on the cell surface or intracellular molecules and the like can be measured. The IFC is a flow cytometer including an imaging unit such as a CCD camera, and is a device capable of acquiring an image of an individual particle in a liquid flowing in a flow cell. For example, the IFC can acquire and quantitatively measure a fluorescence signal, a scattered light signal, a fluorescence image and a bright field image (also referred to as transmitted light image) from each of several 1000 to several 1 million particles, in a short time of several seconds to several minutes. In addition, information of individual particle can be extracted by image processing.

The first information is information on the size of the particles in each measurement sample. The “particles in the measurement sample” refers to a granular object contained in the measurement sample that can be individually measured by a flow cytometer. The particles in the measurement sample may include not only a cell and a cell complex but also a non-cell particle such as lipid particle, debris, and aggregate. The first information on the first measurement sample is used to detect the cell complex in which the T cell and the antigen-presenting cell adhere to each other. The first information on the second measurement sample is used to detect a particle with approximately the same size as the cell complex in which the T cell and the antigen-presenting cell adhere to each other. Examples of the first information include optical information reflecting the particle size. Examples of such optical information include forward scattered light information. The forward scattered light (for example, scattered light near a light receiving angle of 0 to 20 degrees) reflects the particle size. Examples of the forward scattered light information include a pulse peak, pulse width, pulse area, transmittance, Stokes shift, ratio and temporal change of the forward scattered light, a value correlated thereto, and the like.

When the flow cytometer is an IFC, the first information may be, for example, a value related to the size or shape of the particle obtained from the image captured by the IFC. Examples of such a value include an area value, aspect ratio and the like of the particle in the image captured by the IFC. The “area value of the particle” is the number of pixels constituting the image of the particle in the bright field image. Since the cell complex is a complex in which a T cell and an antigen-presenting cell adhere to each other, the area value of the particle is larger than that of a single cell. The “aspect ratio of the particle” is a ratio (major axis/minor axis) of the major axis to the minor axis of an ellipse set to circumscribe the image of the particle in the bright field image. When the value of the aspect ratio of the particle is close to 1, it indicates that the particle is close to a circle, suggesting that the particle is a single cell. On the other hand, when the value of the aspect ratio of the particle is greater than 1, it indicates that the particle is elliptical, suggesting that the particle is a complex of two cells. In a preferred embodiment, the first information is an area value of the particle in the bright field image.

The second information is information on a T cell activation marker. The second information is used to detect a T cell specifically activated by a predetermined antigen. The T cell activation marker may be a marker reflecting the antigen-specific activity of the T cell. The antigen-specific activity refers to a reaction in a T cell that occurs when the T cell specifically recognizes a predetermined antigen. Examples of the T cell activation marker include mitochondria, a predetermined molecule in a T cell, a predetermined substance, and the like. It is known that when a T cell is specifically activated by a predetermined antigen, mitochondria are activated in the T cell, and the amounts of a predetermined molecule and a predetermined substance are changed. Therefore, the second information can be, for example, information on activation of mitochondria in a T cell, information on an amount of a predetermined molecule or a predetermined substance in a T cell, or the like.

The information on activation of mitochondria is, for example, information on magnitude of the membrane potential of mitochondria. It is known that when a T cell is activated by binding to an antigen, the membrane potential of mitochondria in the T cell increases.

Examples of the predetermined molecule in a T cell include a protein molecule expressed in a T cell when the T cell is activated by binding to an antigen, a protein molecule whose expression level or abundance change as compared to that before the T cell is activated, and the like. Examples of such a protein molecule include pERK, NF-_{K}B, NFAT, IFNy, IL-2, and the like. The information on the amount of the predetermined molecule in the T cell is, for example, information indicating expression level of at least one molecule selected from the group consisting of pERK, NF-_{K}B, NFAT, IFNγ, and IL-2. Among them, information indicating abundance of pERK is preferable. When the T cell is activated by binding to an antigen, ERK that is constantly present in the T cell is phosphorylated, and the abundance of pERK is transiently increased.

Examples of the predetermined substance in the T cell include a substance whose intracellular amount changes when the T cell is activated by binding to an antigen as compared to that before activation. Examples of such a substance include intracellular calcium ions and reactive oxygen species (ROS). The information on the amount of the predetermined substance in the T cell is, for example, information indicating an amount of at least one substance selected from the group consisting of calcium ion in the T cell and ROS in the T cell.

In the present embodiment, before acquiring the first information and the second information, it is preferable to label at least one of mitochondria in the T cell, the predetermined molecule and the predetermined substance with a capture body capable of generating an optical signal. The second information can be acquired by detecting the optical signal by a flow cytometer. The type of the optical signal is determined according to the type of a labeling substance described later. Preferably, the optical signal is a fluorescence signal.

When the flow cytometer is an FCM, examples of the second information include information on an optical signal detected by the FCM. In a case where the optical signal is a fluorescence signal, examples of the information on the optical signal include a pulse peak, pulse width, pulse area, transmittance, Stokes shift, ratio and temporal change of fluorescence, a value correlated thereto, and the like.

When the flow cytometer is an IFC, the second information may be, for example, a value based on an optical signal, obtained from an image captured by the IFC. In a case where the optical signal is a fluorescence signal, each pixel constituting an area showing the fluorescence signal has a pixel value corresponding to the fluorescence signal intensity in the fluorescence image of the particle imaged by the IFC. Therefore, the value based on the optical signal, obtained from the image, may be, for example, a value based on a pixel constituting an area showing the fluorescence signal. Examples of such a value include fluorescence intensity, maximum fluorescence intensity, total fluorescence signal intensity, fluorescence signal area value, and the like.

The “fluorescence intensity” is an average value of pixel values of pixels constituting an area showing a fluorescence signal in a fluorescence image of a particle. The “maximum fluorescence intensity” is a maximum value among the pixel values of pixels constituting an area showing a fluorescence signal in a fluorescence image of a particle. The “total fluorescence signal intensity” is a total value of the pixel values of pixels constituting an area showing a fluorescence signal in a fluorescence image of a particle. The “fluorescence signal area value” is the number of pixels constituting an area showing a fluorescence signal in a fluorescence image of a particle. In the present embodiment, a pixel having a pixel value higher than a predetermined pixel value may be extracted from the pixels constituting an area showing a fluorescence signal. Then, a value based on the optical signal may be acquired using the pixel value of the extracted pixel. The predetermined pixel value is not particularly limited, and examples thereof include a pixel value of a region where no cell is present in the fluorescence image.

The capture body capable of generating an optical signal can be appropriately determined according to the object to be labeled. When activated mitochondria or an intracellular substance such as calcium ion or reactive oxygen species (ROS) is labeled, for example, a labeling substance capable of specifically binding to these substances and capable of generating an optical signal may be used. When a protein molecule such as pERK, NF-_{K}B, NFAT, IFNy or IL-2 is labeled, for example, a capture substance that specifically binds to the protein molecule and a labeling substance capable of generating an optical signal may be used. In this case, the protein molecule is labeled by indirectly binding the labeling substance to the protein molecule via the capture body.

When activated mitochondria are labeled, as a capture body capable of generating an optical signal, for example, a fluorescent dye capable of binding depending on the membrane potential of mitochondria can be used. Such a fluorescent dye is known, and examples thereof include JC-1 (5,5′,6,6′-tetrachloro-1,1′,3,3′-tetraethylbenzimidazolylcarbocyanine iodide). A commercially available detection reagent for mitochondrial membrane potential containing such a fluorescent dye may be used. Examples of such a commercially available reagent include MitoTracker (registered trademark) dye (Invitrogen), MitoBright LT dye (DOJINDO LABORATORIES.), and the like.

When an intracellular calcium ion is labeled, for example, a fluorescent calcium indicator can be used as a capture body capable of generating an optical signal. The fluorescent calcium indicator is a fluorescent probe in which a fluorescent dye is coupled to a calcium chelator, and various products are commercially available. Examples of the fluorescent calcium indicator include Fura2, Fluo3, Fluo4, Indo1, Rhod2, and the like. The fluorescent probe may be protected with an acetoxymethyl (AM) group. Protection with an AM group imparts cell permeability to the fluorescent probe.

When intracellular ROS is labeled, as a capture body capable of generating an optical signal, for example, a fluorescent probe that emits fluorescence by a reaction with ROS can be used. Such a fluorescent probe is known, and for example, CellROX (registered trademark) reagent (Thermo Fisher Scientific) is commercially available. The fluorescent probe contained in the reagent does not emit fluorescence in the reduced state, but emits fluorescence by being oxidized by ROS.

Examples of the capture body that specifically binds to a protein molecule include an antibody, an aptamer, a receptor molecule, a ligand molecule, and the like. The antibody is particularly preferable among them. In the present specification, the “antibody” is a concept including an antibody fragment. Examples of the antibody fragment include Fab, F(ab′)2, scFv, and the like.

As the labeling substance capable of generating an optical signal, a fluorescent substance is preferable. The fluorescent substance can be appropriately selected from known fluorescent dyes such as Alexa Fluor (registered trademark), allophycocyanin (APC), FITC, phycoerythrin, rhodamine, and cyanine dyes, known fluorescent proteins such as green fluorescent protein, and the like.

The labeling substance capable of generating an optical signal preferably directly or indirectly binds to a capture body that specifically binds to a protein molecule. Examples of the direct binding between the labeling substance and the capture body include labeling the capture body with the labeling substance. A method for labeling the substance is known per se. For example, the capture body and the labeling substance may be covalently bonded to each other by a crosslinking agent to prepare a labeled capture body. When the capture body is a protein such as an antibody, the capture body may be labeled using a commercially available fluorescence labeling kit or the like. Alternatively, when a labeled antibody against a protein molecule is commercially available, it may be used. The protein molecule is labeled by binding the capture body to which the labeling substance is directly bound to the protein molecule.

Examples of the indirect binding between the labeling substance capable of generating an optical signal and the capture body that specifically binds to a protein molecule include binding between the labeling substance and the capture body via a substance capable of specifically binding to the capture body. In this case, the substance capable of specifically binding to the capture body is preferably labeled with a labeling substance. Examples of the substance capable of specifically binding to the capture body include an antibody, an aptamer, and the like. When the capture body is an antibody, it is preferable to use a labeled antibody that specifically binds to the capture body. In this case, the capture body binds to a protein molecule as a primary antibody, and the labeled antibody that specifically binds to the capture body binds to the capture body bound to the protein molecule as a secondary antibody. Thereby, the labeling substance is indirectly bound to the capture body, and the protein molecule is labeled.

Alternatively, a capture body modified with biotin and its analogs and biotin-binding sites on which a labeling substance is immobilized may be used. In this case, the labeling substance can indirectly bind to the capture body bound to the protein molecule via specific binding between the biotin and its analogs and the biotin-binding sites. The biotin and its analogs include biotin and biotin analogs such as desthiobiotin and oxybiotin. The biotin-binding sites include avidin and avidin analogs such as streptavidin and tamavidin (registered trademark).

When at least one of mitochondria, the predetermined molecule and the predetermined substance in a T cell is labeled, a surface antigen specifically expressed in the T cell may also be labeled. Examples of such a surface antigen include CD4, CD8, and the like. By labeling the surface antigen, a T cell can be selectively detected by a flow cytometer. In this case, the capture body for labeling the surface antigen of the T cell preferably emits an optical signal that can be detected separately from an optical signal emitted by the capture body used for labeling mitochondria or the like.

Information on the optical signal generated from the labeled surface antigen on the T cell is hereinafter referred to as third information. The third information is information for selectively detecting a T cell from the particle measured by the flow cytometer. In a case where the optical signal is a fluorescence signal, examples of the third information include pulse peak, pulse width, pulse area, transmittance, Stokes shift, ratio, temporal change and fluorescence signal area value of fluorescence, a value correlated thereto, and the like.

If necessary, after the cell in the measurement sample is fixed, its cell membrane may be permeabilized. Thereby, the capture body and the labeling substance having no cell permeability can enter the cell, and the molecule in the cell can be labeled. It is also possible to label a cell nucleus. The permeation treatment itself of the cell membrane is known, and examples thereof include a method using a surfactant such as saponin, Triton (registered trademark) X-100, or Tween (registered trademark) 20. In addition, methanol, acetone or ethanol as an organic solvent may be used.

If necessary, cell nucleus of a cell in the measurement sample may be labeled. When the cell membrane is permeabilized, the cell nucleus can be stained with a fluorescent dye capable of staining nucleic acid. Examples of such a fluorescent dye include Hoechst 33342, Hoechst 33258, 4′,6-diamidino-2-phenylindole dihydrochloride (DAPI), and the like.

The flow cytometer is not particularly limited, and commercially available FCM or IFC may be used. Also, the light source of the flow cytometer is not particularly limited, and for example, a light source having a wavelength suitable for exciting the fluorescent substance can be selected appropriately. As the light source, for example, a semiconductor laser light source, an argon laser light source, a He-Ne laser light source, a mercury arc lamp or the like is used. In addition, the bright field light source of the IFC is not particularly limited, and for example, a white laser light source, an LED lamp, a mercury lamp, a xenon lamp or the like is used.

Data of all particles measured by the flow cytometer may be analyzed, or data of some particles may be analyzed. For example, when the particles measured by the flow cytometer are about 1 × 10⁶ particles, data of 50,000 particles may be randomly extracted, and these data may be analyzed. The data of the particles to be analyzed may be selected from, for example, data in focus of an image captured by the IFC. The number of particles to be analyzed is not particularly limited, and may be appropriately determined according to the number of particles contained in the measurement sample. The number of particles to be analyzed may be, for example, 5,000 particles or more and 50,000 particles or less.

A target particle in the first measurement sample is detected based on the first information and the second information on the first measurement sample. Also, a background particle in the second measurement sample is detected based on the first information and the second information on the second measurement sample. In the present specification, the “particle is detected” is a concept including counting particles. In the present embodiment, the number of target particles in the first measurement sample and the number of background particles in the second measurement sample can be acquired.

By the immune reaction described above, a cell complex in which a T cell and an antigen-presenting cell adhere to each other, which contains a T cell activated by a predetermined antigen (hereinafter, also referred to as “cell complex containing an activated T cell”) can be formed in the first measurement sample. The first information and the second information on the first measurement sample are used to detect a cell complex containing an activated T cell. However, as described above, the first measurement sample contains various cells and particles other than the cell complex containing an activated T cell. Therefore, in the extraction of particles according to the first information and the second information, not only a cell complex containing an activated T cell but also a particle other than the complex can be detected. In the present embodiment, among particles detected based on the first information and the second information, a particle that is not a cell complex containing an activated T cell is referred to as “background particle”. The background particle is, for example, a cell complex containing a T cell activated by an antigen other than a predetermined antigen, an aggregate of cells formed by non-specific adhesion, a lipid particle, an aggregate of impurities, a particle that nonspecifically reacts with a labeling substance used for labeling a T cell activity marker, or the like. The “target particle in the first measurement sample” refers to a particle group of a cell complex containing an activated T cell and a background particle.

Since the second measurement sample does not contain an antigen reagent, it is considered that a cell complex containing an activated T cell is not present in the second measurement sample. In the present embodiment, the “background particle in the second measurement sample” refers to a particle group that does not contain a cell complex containing an activated T cell and contains a background particle. Since the first measurement sample and the second measurement sample are prepared from the same biological sample, the background particle in the second measurement sample and the background particle in the first measurement sample are considered to be substantially the same or similar.

As described above, the first information is information related to the particle size. For example, when the first information is information indicated by a numerical value such as forward scattered light intensity, area value or the like of the particle, the value of first information of a cell complex is larger than the value of first information of a single cell. Also, the second information is information on a T cell activation marker. For example, when the second information is information indicated by a numerical value such as a pulse peak of fluorescence or fluorescence intensity acquired from a fluorescence image, the value of second information of an activated T cell is larger than the value of second information of a non-activated T cell. Based on the first information and the second information on the first measurement sample, the measurement data of a particle in the first measurement sample acquired by the flow cytometer is extracted, and the measurement data of the target particle is extracted, whereby the target particle in the first measurement sample can be detected.

In addition, based on the first information and the second information on the second measurement sample, the measurement data of a particle in the second measurement sample acquired by the flow cytometer is extracted, and the measurement data of the background particle is extracted, whereby the background particle in the second measurement sample can be detected.

The first information and the second information may be compared with threshold values corresponding to the first information and the second information respectively, and the target particle and the background particle may be detected based on the comparison result. For example, a particle in which the first information on the first measurement sample is greater than or equal to the first threshold value corresponding to the first information and the second information on the first measurement sample is greater than or equal to the second threshold value corresponding to the second information can be detected as a target particle. Also, a particle in which the first information on the second measurement sample is greater than or equal to the first threshold value and the second information on the second measurement sample is greater than or equal to the second threshold value can be detected as a background particle.

In the present embodiment, the number of target particles and the number of background particles detected based on the first information and the second information for each measurement sample may be converted into the number per predetermined number of particles. This enables comparison of detection results among a plurality of biological samples. The predetermined number of particles is not particularly limited, and can be appropriately determined from, for example, 10,000 particles or more and 1,000,000 particles or less. Here, the “plurality of biological samples” may be a biological sample collected from subject A and a biological sample collected from subject B, or may be a plurality of biological samples collected from the same subject on different days.

The first threshold value corresponding to the first information and the second threshold value corresponding to the second information are not particularly limited, and can be appropriately determined according to the type of each of the first information and the second information. When the first information is an area value of the particle, the first threshold value may be, for example, an area value corresponding to two to three cells of average size. When the first information is an aspect ratio of the particle, the first threshold value may be, for example, a value of the aspect ratio of a cell complex formed by adhesion of two cells of average size. The second threshold value may be determined empirically, for example, by fluorescently labeling mitochondria, a predetermined molecule or a predetermined substance for measurement, using a T cell known to show a specific reaction to a specific antigen, and accumulating data of the second information.

When the flow cytometer is an IFC, after the measurement data of the target particle in the first measurement sample and the background particle in the second measurement sample is extracted based on the first information and the second information, an image of the particle may be visually confirmed. For example, in a case where it is visually confirmed that two cells adhere to each other to form a cell complex in a bright field image, and it is visually confirmed that fluorescent-labeled mitochondria, a spot or an area corresponding to a predetermined molecule or a predetermined substance or the like is present in one cell in a fluorescence image, the cell complex can be said to be a cell complex containing a T cell activated by an antigen. However, it is not known whether the T cell in the cell complex is a T cell activated by a predetermined antigen or a T cell activated by another antigen (for example, a component contained in a medium) by visual confirmation. As described above, the visually confirmed cell complex in the first measurement sample is not necessarily a T cell activated by a predetermined antigen. Therefore, it is preferable to detect the visually confirmed cell complex in the first measurement sample as the target particle. In addition, it is preferable to detect the visually confirmed cell complex in the second measurement sample as the background particle. By visually confirming the image, the number of target particles in the first measurement sample and the number of background particles in the second measurement sample can be counted more accurately.

When the surface antigen specifically expressed in a T cell is labeled, the measurement data of the particles in each measurement sample may be extracted based on the third information before the target particle and the background particle are detected, and the measurement data of a T cell or a particle containing a T cell may be extracted. Thereby, measurement data of a cell other than a T cell, a cell complex not containing a T cell, debris, aggregate and the like can be removed in advance. For example, a particle in which the third information for each measurement sample is greater than or equal to a third threshold value corresponding to the third information can be detected as a T cell or a particle containing a T cell.

When a fluorescence signal area value is acquired as the third information, measurement data of a particle having a fluorescence signal area value for one cell may be extracted after the target particle and the background particle are detected. Thereby, a particle not containing a T cell and a particle containing two or more T cells can be removed from the target particle and the background particle.

When the area value of the particle is acquired as the first information, for example, measurement data of a particle having an area value equal to or less than one cell and a particle having an area value greater than or equal to four cells may be removed from the measurement data of the particles in each measurement sample before the target particle and the background particle are detected. Thereby, measurement data of a single cell, debris, aggregate and the like can be removed in advance.

In the detection method of the present embodiment, a cell complex containing an activated T cell in the first measurement sample is detected based on the detection result of the target particle and the detection result of the background particle. Here, the “cell complex is detected” is a concept including counting cell complexes. As described above, the target particle includes a cell complex containing an activated T cell and a background particle. Also, the background particle in the second measurement sample and the background particle in the first measurement sample are considered to be substantially the same or similar. Therefore, a value obtained by subtracting the number of background particles detected from the second measurement sample from the number of target particles detected from the first measurement sample can be acquired as the number of cell complexes containing an activated T cell in the first measurement sample.

In the present embodiment, the ratio of the number of cell complexes containing an activated T cell in the first measurement sample to the number of particles in the first measurement sample measured by the flow cytometer may be acquired.

In the present embodiment, the number of detected cell complexes containing an activated T cell may be converted into the number per predetermined number of particles. This enables comparison of detection results among a plurality of biological samples. The predetermined number of particles is as described above.

The number of cell complexes containing an activated T cell in the first measurement sample can be an index of specific immune response to a predetermined antigen of a subject. For example, when the number of cell complexes in the first measurement sample is greater than or equal to a predetermined threshold value, it is suggested that the biological sample has been collected from a subject having a specific immune response to the predetermined antigen. In addition, when the number of cell complexes in the first measurement sample is less than the predetermined threshold value, it is suggested that the biological sample has been collected from a subject having no specific immune response to the predetermined antigen. The number of cell complexes containing an activated T cell may be the number per predetermined number of particles.

Another embodiment of the present invention relates to a method for assisting determination of specific immune response to an antigen (hereinafter, also referred to as “method for determining immune response of the present embodiment”). In this method, similarly to those described for the detection method of the present embodiment, the first measurement sample and the second measurement sample are measured by the flow cytometer to acquire first information and second information for each measurement sample. Next, the target particle in the first measurement sample and the background particle in the second measurement sample are detected based on the first information and the second information for each measurement sample. Then, a cell complex containing an activated T cell in the first measurement sample is detected based on the detection results of the target particle and the background particle.

In the method for determining immune response of the present embodiment, whether the biological sample has been collected from a subject having a specific immune response to a predetermined antigen is determined based on the number of detected cell complexes. Specifically, the number of cell complexes containing an activated T cell in the first measurement sample is compared with a predetermined threshold value corresponding to the number of cell complexes containing an activated T cell, and the determination is made based on the comparison result. For example, when the number of cell complexes containing an activated T cell is greater than or equal to the predetermined threshold value, it is determined that the biological sample has been collected from a subject having a specific immune response to a predetermined antigen. In addition, when the number of cell complexes containing an activated T cell is less than the predetermined threshold value, it is determined that the biological sample has been collected from a subject having no specific immune response to a predetermined antigen. The number of cell complexes containing an activated T cell may be the number per predetermined number of particles.

The predetermined threshold value corresponding to the number of cell complexes containing an activated T cell is not particularly limited, and can be appropriately determined. The number of cell complexes containing an activated T cell can also vary depending on the presence or absence of a disease or allergy of the subject, the health condition, and the like. For example, a first measurement sample and a second measurement sample prepared from a biological sample collected from a healthy subject may be measured by a flow cytometer, and a predetermined threshold value may be set based on data of the number of cell complexes containing an activated T cell.

In a further embodiment, a third specimen separated from a biological sample (third aliquot), and a third measurement sample prepared using an immune checkpoint inhibitor and an antigen reagent may be further measured. By detecting a cell complex containing an activated T cell in the third measurement sample, the state of immune tolerance of a subject to a predetermined antigen can be determined. In this embodiment, a third measurement sample prepared by mixing a third specimen that is a part of a biological sample collected from the subject, an antigen reagent, and an immune checkpoint inhibitor in vitro is further used. The third measurement sample can be prepared in the same manner as the first measurement sample by adding an immune checkpoint inhibitor to the third specimen together with an antigen reagent and mixing them. The first specimen, the second specimen and the third specimen are specimens separated from the same biological sample. Then, the third measurement sample is measured by the flow cytometer to acquire first information and second information for the measurement sample. The measurement of the third measurement sample can be performed in the same manner as the measurement of the first measurement sample and the second measurement sample.

The immune checkpoint inhibitor may be a preparation containing at least one of an anti-PD-1 antibody, an anti-CTLA-4 (Cytotoxic T lymphocyte antigen-4) antibody, and an anti-PD-L1 antibody as an active ingredient. Examples of the preparation containing an anti-PD-1 antibody as an active ingredient include nivolumab, pembrolizumab, and the like. Examples of the preparation containing an anti-PD-L1 antibody as an active ingredient include atezolizumab, avelumab, durvalumab, and the like. Examples of the preparation containing an anti-CTLA-4 antibody as an active ingredient include ipilimumab.

When immune tolerance of a subject is determined using the third measurement sample, a control antibody against the immune checkpoint inhibitor may be further added to the first measurement sample. Examples of the control antibody include IgG derived from an unsensitized mammal and the like. In addition, a commercially available control IgG may be used.

In the third measurement sample, the following immune reaction may occur. First, an antigen-presenting cell comes into contact with a predetermined antigen and incorporates the antigen into the cell. Then, the antigen-presenting cell presents all or a part of a predetermined antigen. When a T cell recognizes an antigen presented by an antigen-presenting cell via TCR, the T cell and the antigen-presenting cell adhere to each other to form a cell complex. At this time, antigen-specific activity is generated in the T cell in the cell complex. The antigen-specific activity generated in the T cell is reflected in a T cell activation marker. On the other hand, when the T cell is in a state of immune tolerance to the antigen, antigen-specific activity does not occur in the T cell in the cell complex. However, since the immune checkpoint inhibitor has been added to the third measurement sample, the state of immune tolerance of T cell can be released. Therefore, when there is an effect of the immune checkpoint inhibitor, the third measurement sample may contain more cell complexes containing an activated T cell than the first measurement sample. The increase in the cell complex containing an activated T cell reflects the state of immune tolerance to a predetermined antigen of a subject.

In a further embodiment, a target particle in the third measurement sample is detected based on the first information and the second information on the third measurement sample. The “target particle in the third measurement sample” refers to a particle group of a cell complex containing an activated T cell and a background particle. As described above, due to the effect of the immune checkpoint inhibitor, the target particle in the third measurement sample may contain more cell complexes containing an activated T cell than the target particle in the first measurement sample.

In a further embodiment, the first information and the second information on the third measurement sample may be compared with the first threshold value and the second threshold value, and the target particle may be detected based on the comparison result. For example, a particle in which the first information on the third measurement sample is greater than or equal to the first threshold value and the second information on the third measurement sample is greater than or equal to the second threshold value can be detected as a target particle in the third measurement sample. When the flow cytometer is an IFC, after the measurement data of the target particle in the third measurement sample is extracted based on the first information and the second information, an image of the particle may be visually confirmed. The visual confirmation of the images acquired by the IFC is as described above.

In a further embodiment, a cell complex containing an activated T cell in the third measurement sample is detected based on the detection result of the target particle of the third measurement sample and the detection result of the background particle of the second measurement sample. Specifically, a value obtained by subtracting the number of background particles detected from the second measurement sample from the number of target particles detected from the third measurement sample can be acquired as the number of cell complexes containing an activated T cell in the third measurement sample. The number of target particles in the third measurement sample may be the number per predetermined number of particles. Then, by comparing the number of cell complexes containing an activated T cell between the first measurement sample and the third measurement sample, the state of immune tolerance of a subject to a predetermined antigen can be determined. For example, first, a value obtained by subtracting the number of cell complexes containing an activated T cell in the first measurement sample from the number of cell complexes containing an activated T cell in the third measurement sample is acquired. The number of cell complexes containing an activated T cell in each measurement sample may be the number per predetermined number of particles. Then, the acquired difference value is compared with a predetermined threshold value corresponding to the difference value. When the acquired difference value is greater than or equal to the predetermined threshold value, it is suggested that the biological sample has been collected from a subject who is in a state of immune tolerance to a predetermined antigen. In addition, when the acquired difference value is less than the predetermined threshold value, it is suggested that the biological sample has been collected from a subject who is not in a state of immune tolerance to a predetermined antigen.

A further embodiment of the present invention relates to a method for assisting determination of immune tolerance of a subject (hereinafter, also referred to as “method for determining immune tolerance of the present embodiment”). In this method, similarly to those described for the detection method of the present embodiment, the first measurement sample, the second measurement sample and the third measurement sample are measured by the flow cytometer to acquire first information and second information for each measurement sample. Next, the target particle in the first measurement sample, the background particle in the second measurement sample and the target particle in the third measurement sample are detected based on the first information and the second information for each measurement sample. Then, a cell complex containing an activated T cell in the first measurement sample is detected based on the detection results of the target particle in the first measurement sample and the background particle in the second measurement sample. In addition, a cell complex containing an activated T cell in the third measurement sample is detected based on the detection results of the target particle in the third measurement sample and the background particle in the second measurement sample.

In the method for determining immune tolerance of the present embodiment, whether a biological sample has been collected from a subject who is in a state of immune tolerance to a predetermined antigen is determined based on the number of cell complexes containing an activated T cell in the first measurement sample and the third measurement sample. First, a value obtained by subtracting the number of cell complexes containing an activated T cell in the first measurement sample from the number of cell complexes containing an activated T cell in the third measurement sample is acquired. The number of cell complexes containing an activated T cell in each measurement sample may be the number per predetermined number of particles. Then, the acquired difference value is compared with a predetermined threshold value corresponding to the difference value. When the acquired difference value is greater than or equal to the predetermined threshold value, it is determined that the biological sample has been collected from a subject who is in a state of immune tolerance to a predetermined antigen. In addition, when the acquired difference value is less than the predetermined threshold value, it is determined that the biological sample has been collected from a subject who is not in a state of immune tolerance to a predetermined antigen.

The predetermined threshold value corresponding to the difference value is not particularly limited, and can be appropriately determined. For example, a first measurement sample, a second measurement sample and a third measurement sample prepared from a biological sample collected from a subject known to be in a state of immune tolerance to a predetermined antigen may be measured by a flow cytometer, and a predetermined threshold value may be set based on data of the number of cell complexes containing an activated T cell.

A reagent kit of the present embodiment can be used in the method for detecting an antigen-specific activated T cell, the method for assisting determination of immune response of a subject, and the method for assisting determination of immune tolerance of a subject. The reagent kit of the present embodiment contains a capture body capable of generating an optical signal for labeling at least one selected from the group consisting of mitochondria, pERK, NF-κB, NFAT, IFNγ, IL-2, calcium ions, and ROS in a T cell.

In one embodiment, a container containing one reagent selected from a reagent containing a labeling substance that specifically binds to activated mitochondria, a reagent containing a labeling substance that specifically binds to pERK, a reagent containing a labeling substance that specifically binds to NF-κB, a reagent containing a labeling substance that specifically binds to NFAT, a reagent containing a labeling substance that specifically binds to IFNy, a reagent containing a labeling substance that specifically binds to IL-2, a reagent containing a labeling substance that specifically binds to a calcium ion, and a reagent containing a labeling substance that specifically binds to ROS may be packed in a box and the like to be provided to a user as a reagent kit. The box may contain an attached document. Compositions, usage, storage method, etc. of the reagents may be described in the attached document.

Examples of the labeling substance that specifically binds to activated mitochondria include fluorescent dyes capable of binding depending on the membrane potential of mitochondria. Examples of the labeling substance that specifically binds to pERK, NF-κB, NFAT, IFNy or IL-2 include a capture body that specifically binds to each protein molecule and is labeled with a fluorescent substance. Examples of the labeling substance that specifically binds to a calcium ion include a fluorescent calcium indicator. Examples of the labeling substance that specifically binds to ROS include a fluorescent probe that emits fluorescence by a reaction with ROS. These labeling substances are the same as those described for the method of the present embodiment.

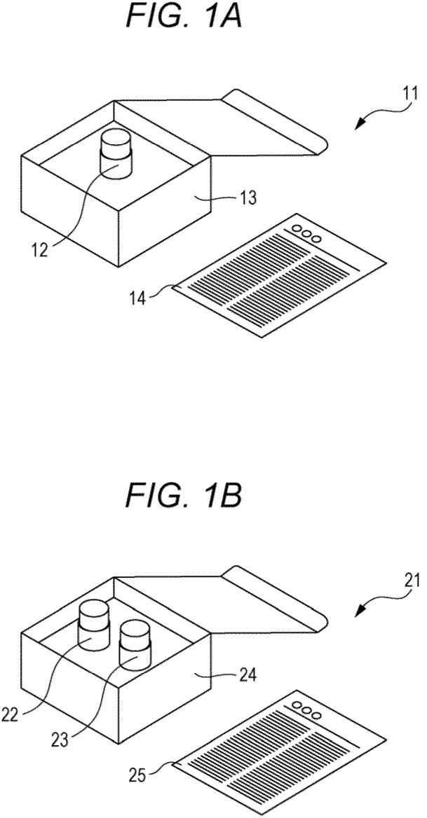

FIG. 1A shows an example of the reagent kit. In FIGS. 1A, 11 denotes a reagent kit, 12 denotes a container containing a reagent containing a labeling substance that specifically binds to activated mitochondria, 13 denotes a packing box, and 14 denotes an attached document. The reagent kit of this example may be provided with a reagent containing another labeling substance, instead of the reagent containing a labeling substance that specifically binds to activated mitochondria.

In a further embodiment, as a labeling substance that specifically binds to pERK, NF-κB, NFAT, IFNγ or IL-2, for example, a combination of an antibody that specifically binds to each protein molecule and a fluorescently labeled secondary antibody that specifically binds to the antibody may be used. FIG. 1B shows an example of the reagent kit. In FIG. 1B, 21 denotes a reagent kit, 22 denotes a first container containing a reagent containing an antibody that specifically binds to pERK, 23 denotes a second container containing a reagent containing a fluorescently labeled secondary antibody that specifically binds to an antibody in the reagent contained in the first container, 24 denotes a packing box, and 25 denotes an attached document. The reagent kit of this example may be provided with a reagent containing an antibody that specifically binds to another protein molecule, instead of the reagent containing an antibody that specifically binds to pERK.

An analysis device of measurement data of the flow cytometer of the present embodiment (hereinafter, also referred to as “analysis device of the present embodiment”) will be described below with reference to the drawings, but the present invention is not limited to this description.

### <Embodiment 1 of Cell Analyzer>

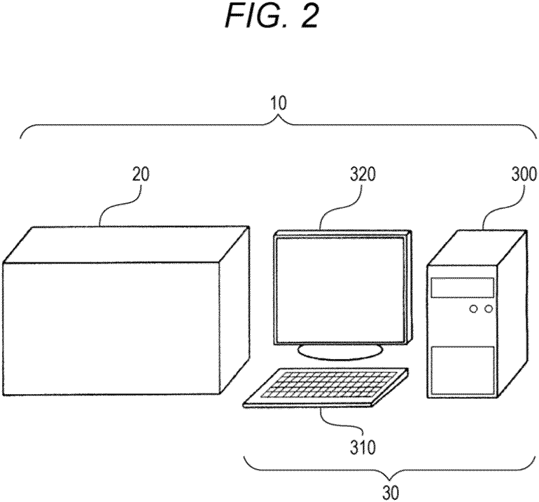

Referring to FIG. 2, a cell analyzer 10 includes a measuring device 20 and an analysis device 30. The analysis device 30 includes a main body 300, an input unit 310, and a display unit 320. In the present embodiment, the measuring device 20 is an IFC, and optically measures an individual particle in a measurement sample by a flow cytometry method to capture an image of each particle. The analysis device 30 analyzes the measurement data acquired by the measuring device 20. The analysis device 30 displays the analysis result on a display unit 320. The present embodiment is not limited to this example, and may be, for example, a device in which the measuring device 20 and the analysis device 30 are integrally configured.

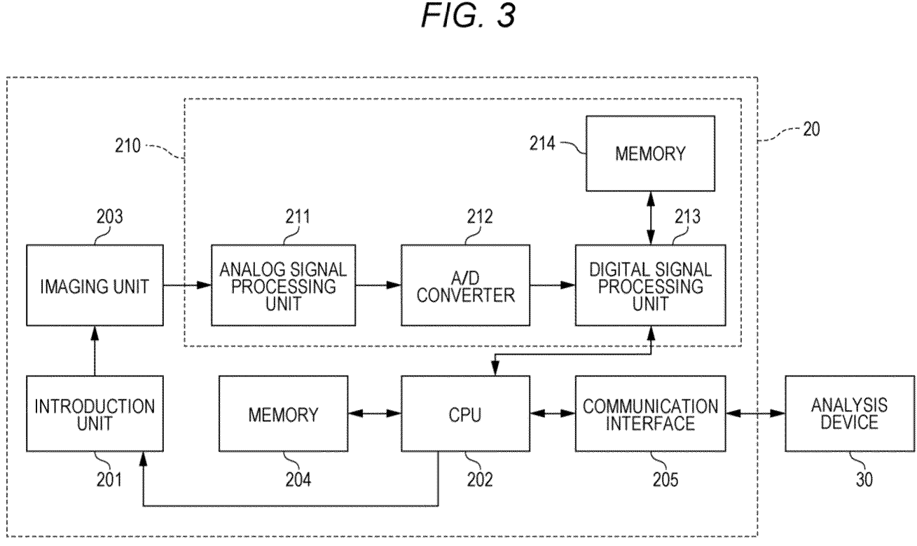

Referring to FIG. 3, the measuring device 20 has an introduction unit 201, a CPU 202, an imaging unit 203, a memory 204, a communication interface 205, and a signal processing unit 210. The signal processing unit 210 includes an analog signal processing unit 211, an A/D converter 212, a digital signal processing unit 213, and a memory 214.

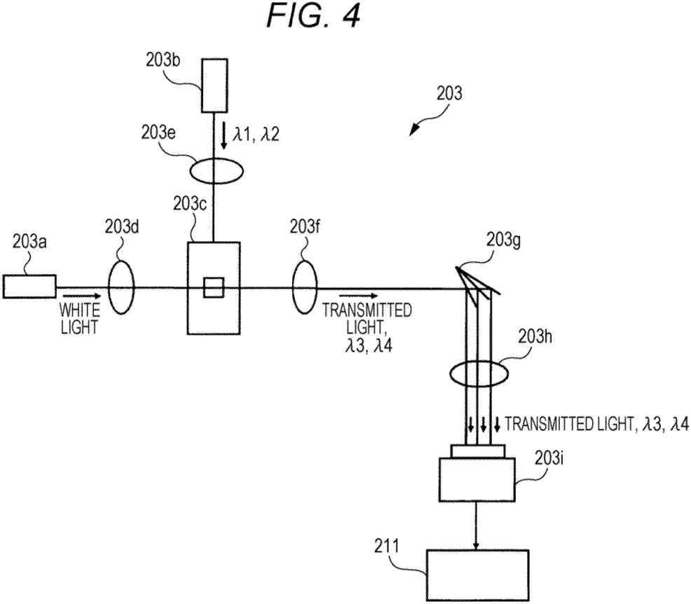

The introduction unit 201 includes a container and a pump (not shown). The first measurement sample in the container is supplied to a flow cell 203c (see FIG. 4) of the imaging unit 203 together with a sheath liquid by a pump. The introduction unit 201 supplies the first measurement sample to the imaging unit 203 in accordance with an instruction from the CPU 202. The second measurement sample is also supplied to the imaging unit 203 in the same manner as the first measurement sample. In this example, the first measurement sample is prepared as follows. PBMC and an antigen reagent are mixed and incubated in vitro. MitoTracker (registered trademark) Deep Red (hereinafter, also referred to as “MTDR”) is added thereto and further incubated. After completion of the incubation, the cell is fixed and washed, followed by being stained with a FITC-labeled anti-CD4 antibody to obtain a first measurement sample. Also, the second measurement sample can be prepared in the same manner as the first measurement sample except that the aqueous solvent used for the reagent is used instead of the antigen reagent. In this example, when a T cell is activated by a predetermined antigen, activated mitochondria in the T cell are labeled with MTDR in a cell complex of the T cell and an antigen-presenting cell. In addition, the T cell is fluorescently stained with the FITC-labeled anti-CD4 antibody. However, the present invention is not limited to this example. Instead of labeling activated mitochondria, for example, pERK in the T cell may be fluorescently labeled. In this case, the dye of the fluorescent label of the CD4 antibody can be changed so as to be distinguishable from the fluorescence of the fluorescent label used in the detection of pERK.

The imaging unit 203 irradiates each measurement sample supplied from the introduction unit 201 with a laser beam, images a particle containing a labeled T cell. The imaging unit 203 outputs image information of the generated bright field image and fluorescence image as an electric signal to the analog signal processing unit 211. The analog signal processing unit 211 amplifies the electric signal outputted from the imaging unit 203 by an amplifier circuit that amplifies the electric signal. The analog signal processing unit 211 outputs the electric signal to the A/D converter 212.

The A/D converter 212 converts the electric signal amplified by the analog signal processing unit 211 into a digital signal. The A/D converter 212 outputs the digital signal to the digital signal processing unit 213. In accordance with an instruction from the CPU 202, the digital signal processing unit 213 performs predetermined signal processing on the digital signal outputted from the A/D converter 212, and measurement data is generated. The generated measurement data is stored in the memory 214.

The measurement data stored in the memory 214 includes a bright field image and a fluorescence image based on each of transmitted light and fluorescence generated when an individual particle in each measurement sample passed through the flow cell 203c. The CPU 202 outputs the measurement data stored in the memory 214 to the communication interface 205. The CPU 202 receives a control signal from the analysis device 30 via the communication interface 205. The CPU 202 controls each unit of the measuring device 20 according to the control signal. The communication interface 205 transmits the measurement data outputted from the memory 214 to the analysis device 30. The communication interface 205 receives the control signal outputted from the analysis device 30. The memory 204 is used as a work area of the CPU 202.

Referring to FIG. 4, the imaging unit 203 includes light sources 203a and 203b, a flow cell 203c, condenser lenses 203d and 203e, an objective lens 203f, an optical unit 203g, a condenser lens 203h, and a camera 203i.

In this embodiment, the light source 203a is a semiconductor laser light source. The light irradiated from the light source 203a is a white laser beam. The condenser lens 203d collects the light irradiated from the light source 203a. The condenser lens 203d guides the collected light to the sample in the flow cell 203c. The white laser beam irradiated from the light source 203a is irradiated onto the individual particle passing through the inside of the flow cell 203c, and transmitted light is generated from each particle.

The light source 203b is a semiconductor laser light source. Preferably, the light source 203b is a semiconductor laser light source that can emit laser beams with a plurality of different wavelengths. In FIG. 4, the light irradiated from the light source 203b is laser beams of wavelengths λ1 and λ2. In Embodiment 1, the wavelength λ1 is, for example, about 645 nm, and the wavelength λ2 is, for example, about 490 nm. The condenser lens 203e collects the light irradiated from the light source 203b. The condenser lens 203e guides the collected light to the measurement sample in the flow cell 203c. The light of wavelengths λ1 and λ2 irradiated from the light source 203b is irradiated onto the individual particle passing through the inside of the flow cell 203c. For example, when a T cell labeled with MTDR and FITC-labeled anti-CD4 antibody is irradiated with a laser beam, fluorescence with a wavelength λ3 (for example, about 665 nm) is generated from MTDR, and fluorescence with a wavelength λ4 (for example, about 515 nm) is generated from FITC.

The objective lens 203f collects the transmitted light and the fluorescence of wavelengths λ3 and λ4. The optical unit 203g has a configuration in which a plurality of dichroic mirrors are combined. In the present embodiment, the three dichroic mirrors of the optical unit 203g reflect the transmitted light and the fluorescence of wavelengths λ3 and λ4 at mutually different angles and are separated on a light receiving surface of the camera 203i described later. The condenser lens 203h collects the transmitted light and the fluorescence of wavelengths λ3 and λ4. The camera 203i receives the transmitted light and the fluorescence of wavelengths λ3 and λ4. The camera 203i outputs image information of the particle in the flow cell 203c as an electric signal to the analog signal processing unit 211. The camera 203i may be a TDI (Time Delay Integration)-CCD camera. By using the TDI-CCD camera, the particle can be imaged with high sensitivity.

The camera 203i receives the transmitted light and the fluorescence of wavelengths λ3 and λ4 in different light receiving regions on the light receiving surface. The light receiving surface is a light receiving surface of an image sensor such as CCD and CMOS disposed in the camera 203i. The position of the light irradiated to the light receiving surface moves in each light receiving region in accordance with movement of the particle in the flow cell 203c. As described above, since the three lights are separated on the light receiving surface by the optical unit 203g, the CPU 202 can extract signals corresponding to each light from the image signal outputted by the camera 203i.

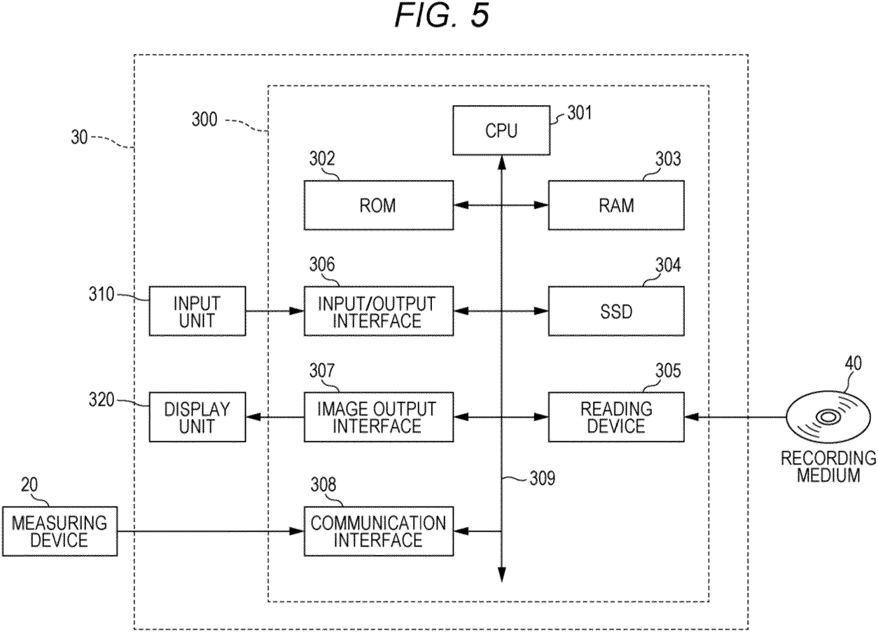

Referring to FIG. 5, the analysis device 30 includes a main body 300, an input unit 310, and a display unit 320. The main body 300 includes a CPU 301, a ROM 302, a RAM 303, a solid state drive (SSD) 304, a reading device 305, an input/output interface 306, an image output interface 307, and a communication interface 308. These are data-communicably connected by a bus 309. Also, the measuring device 20 is communicably connected to the analysis device 30 via a communication interface 308.

The CPU 301 executes a computer program stored in the ROM 302 and a computer program loaded in the RAM 303. The computer program stored in ROM 302 includes a basic input output system (BIOS). The RAM 303 is used for reading the computer program stored in the ROM 302 and the SSD 304. The RAM 303 is also used as a work area of the CPU 301 when executing these computer programs.

In the SSD 304, various computer programs to be executed by the CPU 301, such as operating systems (OS) and application programs, and data used for executing the computer programs are stored. Also, the measurement data received from the measuring device 20 is stored in the SSD 304. In the present embodiment, a hard disk drive may be used instead of the SSD.

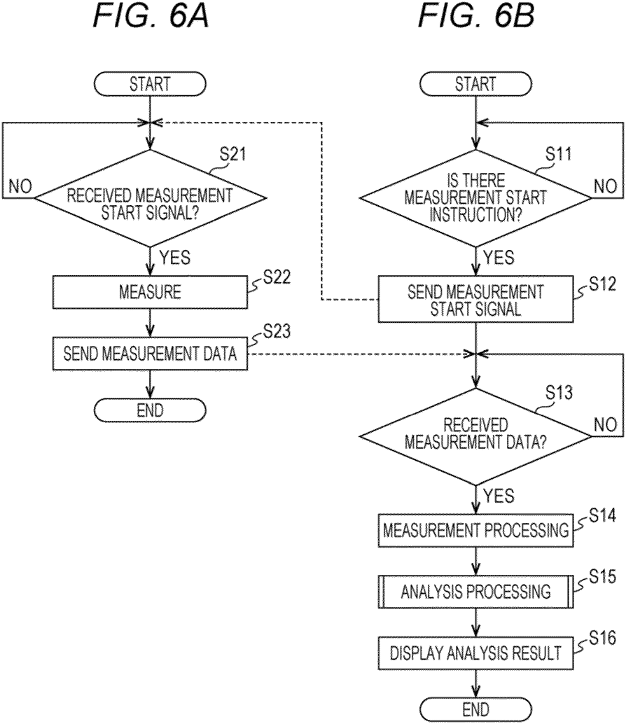

The SSD 304 stores a program for measuring parameters for analysis including the number of particles contained in the measurement sample, the particle size, the fluorescence intensity and the like, based on the measurement data, to analyze the measurement sample, and a display program for displaying the analysis result on the display unit 320. Also, the SSD 304 stores various predetermined threshold values. By storing the above programs in the SSD 304, analysis processing and display processing described later are performed. That is, the CPU 301 is provided with a function of executing the processing of FIG. 6B described later by these programs.

The reading device 305 includes, for example, a flexible disk drive, a CD-ROM drive, a DVD-ROM drive, a USB port, an SD card reader, a CF card reader, a memory stick reader, and the like. The reading device 305 can read a computer program and data recorded on a recording medium 40. The recording medium 40 is a portable recording medium that can be read by the reading device 305, and examples thereof include a flexible disk, a CD, a DVD, a USB flash drive, an SD card, a CF card, a memory stick, and the like.

The input unit 310 including a mouse, a keyboard and the like is connected to the input/output interface 306, and the user inputs an instruction to the analysis device 30, using the input unit 310. The image output interface 307 is an interface conforming to a predetermined standard. The predetermined standard may be, for example, D-Sub, DVI-I, DVI-D, HDMI (registered trademark), DisplayPort, or the like. The image output interface 307 is connected to the display unit 320 including display and the like via a cable corresponding to the standard. Thereby, a video signal corresponding to an image data is output to the display unit 320. The display unit 320 displays an image based on the inputted video signal.

The communication interface 308 is a wired interface conforming to a standard such as an Ethernet (registered trademark) interface. The analysis device 30 can receive the measurement data transmitted from the measuring device 20 through the communication interface 308. The received measurement data is stored in the SSD 304.

Control processing by the CPU 202 of the measuring device 20 and the CPU 301 of the analysis device 30 will be described with reference to FIG. 6. FIG. 6A is a flowchart showing control processing by the CPU 202 of the measuring device 20, and FIG. 6B is a flowchart showing control processing by the CPU 301 of the analysis device 30.

Regarding the control processing of the CPU 301 of the analysis device 30, refer to FIG. 6B. In step S11, when a measurement start instruction is given from the user via the input unit 310, the CPU 301 transmits a measurement start signal to the measuring device 20 in step S12. Then, in step S13, the CPU 301 determines whether the measurement data has been received. In step S13, when the measurement data has not been received, the processing is on standby.

Regarding the control processing of the CPU 202 of the measuring device 20, refer to FIG. 6A. Upon receiving the measurement start signal from the analysis device 30 in step S21, the CPU 202 performs measurement of the first measurement sample in step S22. In the measurement processing in step S22, a first measurement sample containing a labeled T cell is supplied together with a sheath liquid from the introduction unit 201 to the flow cell 203c, and a flow of the first measurement sample enclosed in the sheath liquid is formed in the flow cell 203c. Laser beams from the light sources 203a and 203b are irradiated onto the formed flow, and a beam spot is formed on the flow cell 203c. When the individual particle in the first measurement sample passes through the beam spot, transmitted light and fluorescence are generated. The generated transmitted light and fluorescence are imaged by the camera 203i, respectively, and converted into electric signals.

The electric signals are converted into digital signals by the A/D converter 212, and subjected to signal processing by the digital signal processing unit 213. Thereby, measurement data including a bright field image and a fluorescence image is obtained for each complex that has passed through the flow cell 203c. The measurement data is stored in the memory 214. After the measurement of the first measurement sample is completed, the second measurement sample is measured. The measurement of the second measurement sample is performed in the same manner as the measurement of the first measurement sample. Upon completion of the measurement, the CPU 202 transmits the measurement data generated by the measurement processing to the analysis device 30 in step S23, and the CPU 202 ends the processing.

Referring to in FIG. 6B, upon receiving the measurement data from the measuring device 20 in step S13, the CPU 301 of the analysis device 30 stores the measurement data in the SSD 304. In step S14, the CPU 301 performs measurement processing based on the measurement data to acquire the parameters for analysis, and the CPU 301 stores the parameters for analysis in the SSD 304. In step S15, the CPU 301 performs analysis processing using the acquired parameters for analysis. In step S16, the CPU 301 stores the analysis result acquired in step S15 in the SSD 304. In addition, the CPU 301 displays the analysis result on the display unit 320, and the CPU 301 ends the processing.

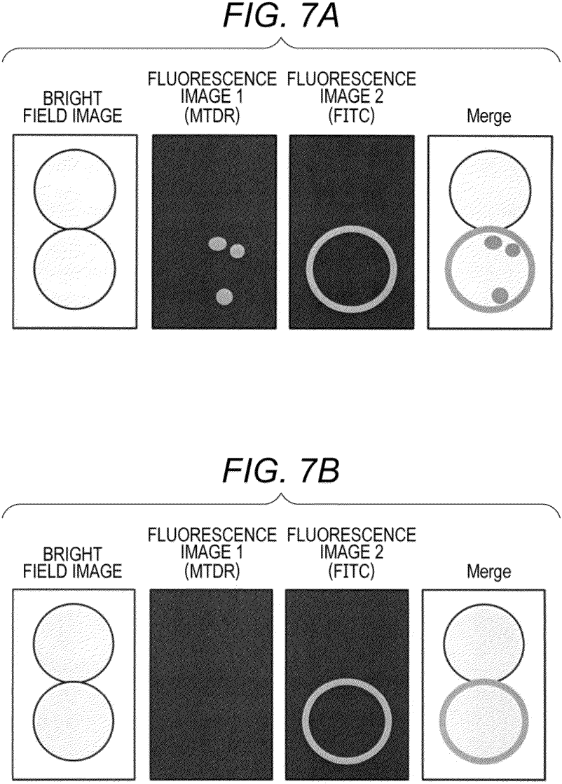

An image captured by the measuring device 20 will be described with reference to FIGS. 7A and 7B. In the drawing, the bright field image is an image based on transmitted light. Fluorescence images 1 and 2 are images based on fluorescence emitted from MTDR and FITC, respectively. When the imaged particle is a cell complex of a T cell and an antigen-presenting cell, cell doublets are seen as shown in the bright field images of FIGS. 7A and 7B. In addition, CD4 on the surface of a T cell is stained with FITC as shown in the fluorescence image 2. When the T cell is activated by a predetermined antigen presented by an antigen-presenting cell, activated mitochondria labeled with MTDR are seen as shown in the fluorescence image 1 of FIG. 7A. On the other hand, when the T cell is not activated by a predetermined antigen, mitochondria are not activated and are not labeled with MTDR, and thus nothing is displayed as in the fluorescence image 1 of FIG. 7B. The image shown in FIG. 7B can be captured when a predetermined antigen is not recognized by a T cell or a T cell is immune tolerance to the antigen.

Referring to in FIG. 6, in step S14, the CPU 301 performs measurement processing based on the bright field images and fluorescence images of all the particles imaged in step S22 to acquire the parameters for analysis. The parameters for analysis are not particularly limited, and examples thereof include an area value (the number of pixels) of the particle, an aspect ratio of the particle, a fluorescence intensity, a maximum fluorescence intensity, and the like. The parameters for analysis are the same as those described for the method of the present embodiment above.

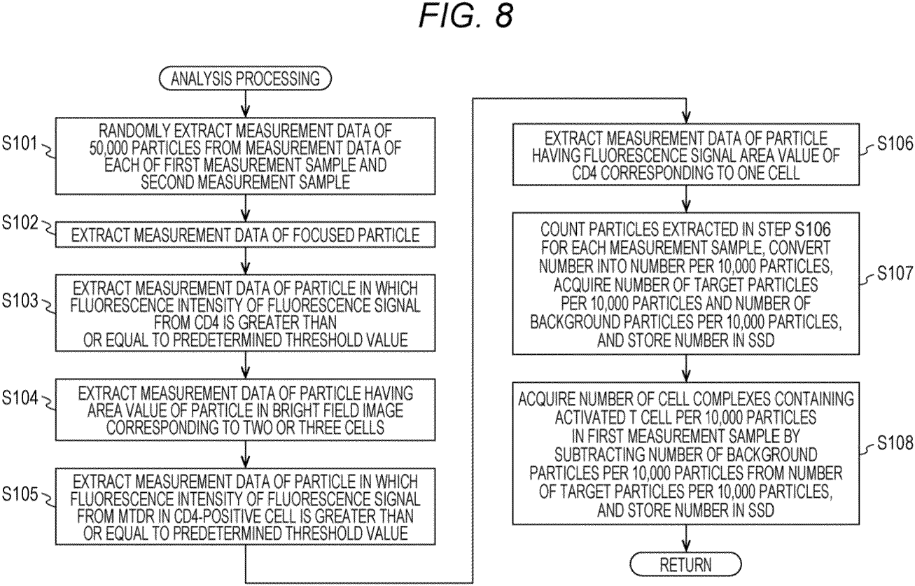

The flow of the analysis processing in step S15 of FIG. 6 will be described with reference to FIG. 8. However, the present invention is not limited to this analysis processing. In step S101, the CPU 301 randomly extracts measurement data of tens of thousands of particles (for example, 50,000 particles) from the measurement data of each of the first measurement sample and the second measurement sample acquired in step S13 of FIG. 6. The random extraction method is not particularly limited. In step S102, the CPU 301 extracts measurement data of the particle focused on the image from the measurement data extracted in step S101 for each measurement sample. In step S103, the CPU 301 extracts measurement data of a particle in which the fluorescence intensity of the fluorescence signal from FITC is greater than or equal to a predetermined threshold value from the measurement data extracted in step S102 for each measurement sample, using the parameters for analysis acquired in step S14 of FIG. 6. Thereby, measurement data of a CD4-positive cell and a particle containing the cell can be extracted for each measurement sample.

In step S104, the CPU 301 extracts measurement data of a particle having an area value of the particle in the bright field image corresponding to two to three cells from the measurement data extracted in step S103 for each measurement sample, using the parameters for analysis. The particle having an area value of the particle corresponding to two to three cells may be a cell complex to which two to three cells adhere. Thereby, measurement data of a cell complex containing a CD4-positive cell can be extracted. In step S105, the CPU 301 extracts measurement data of a particle in which the fluorescence intensity of the fluorescence signal from MTDR is greater than or equal to a predetermined threshold value from the measurement data extracted in step S104 for each measurement sample. A particle in which the fluorescence intensity of the fluorescence signal from MTDR is greater than or equal to a predetermined threshold value may be a particle containing an activated T cell. Thereby, data of the cell complex containing a CD4-positive activated T cell can be extracted. In step S106, the CPU 301 extracts measurement data of a particle having a fluorescence signal area value of FITC corresponding to one cell from the measurement data extracted in step S105 for each measurement sample. The particle having a fluorescence signal area value of FITC corresponding to one cell may be a particle containing only one activated T cell. Thereby, data of the cell complex containing only one CD4-positive activated T cell can be extracted.

By the extraction from step S102 to step S106, measurement data of a target particle is extracted from the measurement data of the first measurement sample, and measurement data of a background particle is extracted from the measurement data of the second measurement sample. In step S107, the CPU 301 counts the particles extracted in step S106 for each measurement sample, the CPU 301 converts the number into the number per focused 10,000 particles, and the CPU 301 acquires the number of target particles per 10,000 particles and the number of background particles per 10,000 particles. The CPU 301 stores the number acquired in step S107 in the SSD 304. In step S108, the number of cell complexes containing an activated T cell per 10,000 particles in the first measurement sample is acquired by subtracting the number of background particles per 10,000 particles from the number of target particles per 10,000 particles. The CPU 301 may store the number acquired in step S108 in the SSD 304. The CPU 301 may display the number as an analysis result on the display unit 320.

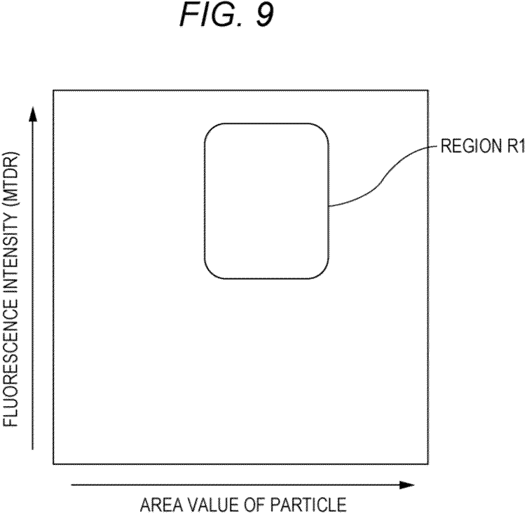

In step S105, the CPU 301 may create a scattergram in which the area value of the particle is taken on X-axis and the fluorescence intensity of the fluorescence signal from MTDR is taken on Y-axis. In the scattergram, a dot linked to the measurement data of individual particle measured in step S22 of FIG. 6 is displayed. When a user designates the dot displayed on the scattergram, the CPU 301 reads from the SSD 304 the measurement data (bright field image and/or fluorescence image) of the particle corresponding to the dot, and the CPU 301 displays it on the display unit 320. Referring to FIG. 9, region R1 is a region in which the area value of the particle (X axis) is gated in a range of two to three cells, and the fluorescence intensity (Y axis) is gated in a predetermined range reflecting the presence of activated mitochondria. For example, in the scattergram for the first measurement sample, a dot of the target particle may be displayed in the region R1. Also, in the scattergram for the second measurement sample, a dot of the background particle may be displayed in the region R1.

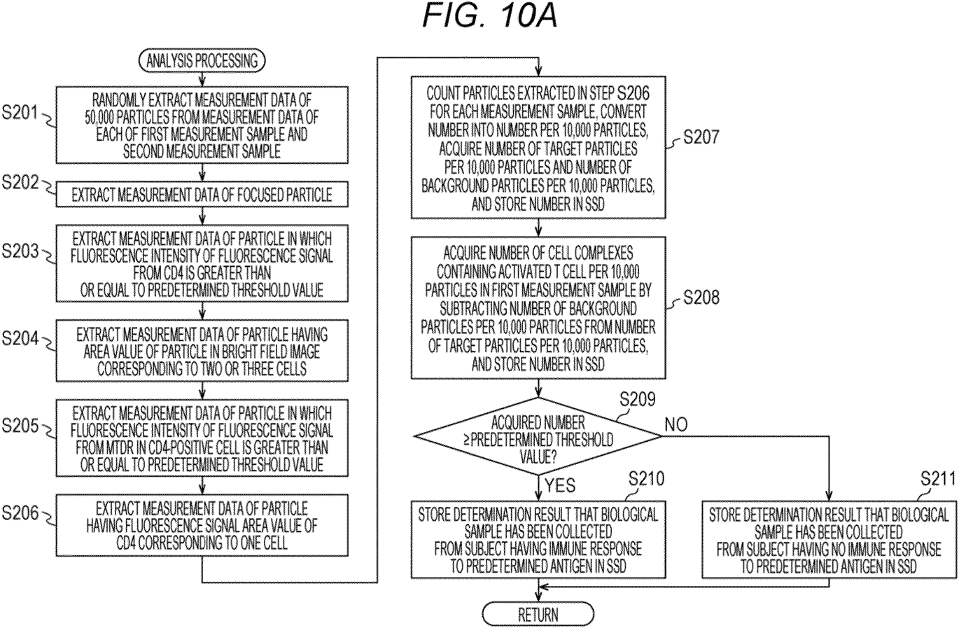

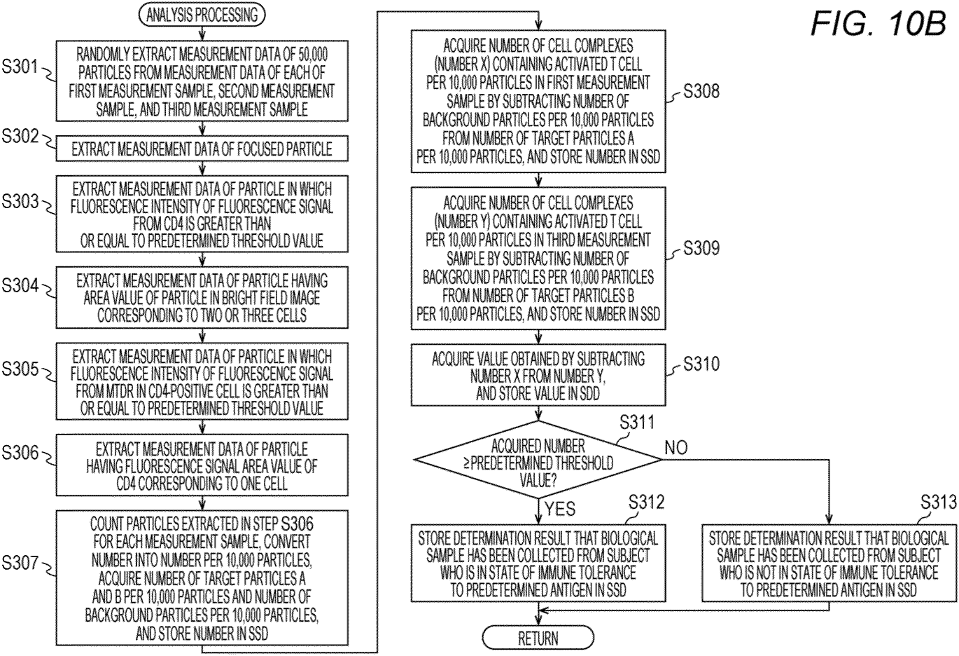

Referring to FIG. 10A, a flow in the case of determining specific immune response to an antigen on the basis of the number of cell complexes containing an activated T cell per 10,000 particles will be described. However, the present invention is not limited to this analysis processing. In step S201, the CPU 301 randomly extracts measurement data of tens of thousands (for example, 50,000) of particles from the measurement data of each of the first measurement sample and the second measurement sample acquired in step S13 of FIG. 6. In step S202, the CPU 301 extracts measurement data of the particle focused on the image from the measurement data extracted in step S201 for each measurement sample. In step S203, the CPU 301 extracts measurement data of a particle in which the fluorescence intensity of the fluorescence signal from FITC (CD4) is greater than or equal to a predetermined threshold value from the measurement data extracted in step S202 for each measurement sample in the same manner as in step S103 of FIG. 8. In step S204, the CPU 301 extracts measurement data of a particle having an area value of the particle corresponding to two to three cells from the measurement data extracted in step S203 for each measurement sample, in the same manner as in step S104 of FIG. 8. In step S205, the CPU 301 extracts measurement data of a particle in which the fluorescence intensity of the fluorescence signal from MTDR is greater than or equal to a predetermined threshold value from the measurement data extracted in step S204 for each measurement sample in the same manner as in step S105 of FIG. 8. In step S206, the CPU 301 extracts measurement data of a particle having a fluorescence signal area value of FITC corresponding to one cell from the measurement data extracted in step S205 for each measurement sample.

In step S207, the CPU 301 counts the particles extracted in step S206 for each measurement sample, the CPU 301 converts the number into the number per focused 10,000 particles, and the CPU 301 acquires the number of target particles per 10,000 particles and the number of background particles per 10,000 particles. The CPU 301 stores the number acquired in step S207 in the SSD 304. In step S208, the number of cell complexes containing an activated T cell per 10,000 particles in the first measurement sample is acquired by subtracting the number of background particles per 10,000 particles from the number of target particles per 10,000 particles. The CPU 301 stores the number acquired in step S208 in the SSD 304.

In step S209, the CPU 301 compares the number acquired in step S208 with the predetermined threshold value stored in the SSD 304. When the acquired number is greater than or equal to the predetermined threshold value, the process proceeds to step S210. In step S210, the CPU 301 stores a determination result that the biological sample has been collected from a subject having an immune response to a predetermined antigen in the SSD 304. In step S209, when the acquired number is less than the predetermined threshold value, the process proceeds to step S211. In step S211, the CPU 301 stores a determination result that the biological sample has been collected from a subject having no immune response to a predetermined antigen in the SSD 304. The CPU 301 may display the determination result of step S210 or S211 on the display unit 320.

Referring to FIG. 10B, a flow in the case of determining immune tolerance to an antigen on the basis of the number of cell complexes containing an activated T cell per 10,000 particles will be described. However, the present invention is not limited to this analysis processing. In step S301, the CPU 301 randomly extracts measurement data of tens of thousands of particles (for example, 50,000 particles) from the measurement data of each of the first measurement sample, the second measurement sample and the third measurement sample acquired in step S13 of FIG. 6. In step S302, the CPU 301 extracts measurement data of the particle focused on the image from the measurement data extracted in step S301 for each measurement sample. In step S303, the CPU 301 extracts measurement data of a particle in which the fluorescence intensity of the fluorescence signal from FITC (CD4) is greater than or equal to a predetermined threshold value from the measurement data extracted in step S302 for each measurement sample in the same manner as in step S103 of FIG. 8. In step S304, the CPU 301 extracts measurement data of a particle having an area value of the particle corresponding to two to three cells from the measurement data extracted in step S303 for each measurement sample, in the same manner as in step S104 of FIG. 8. In step S305, the CPU 301 extracts measurement data of a particle in which the fluorescence intensity of the fluorescence signal from MTDR is greater than or equal to a predetermined threshold value from the measurement data extracted in step S304 for each measurement sample in the same manner as in step S105 of FIG. 8. In step S306, the CPU 301 extracts measurement data of a particle having a fluorescence signal area value of FITC corresponding to one cell from the measurement data extracted in step S305 for each measurement sample.

In step S307, the CPU 301 counts the particles extracted in step S306 for each measurement sample, the CPU 301 converts the number into the number per focused 10,000 particles, and the CPU 301 acquires the number of target particles A and B per 10,000 particles and the number of background particles per 10,000 particles. Here, the “target particle A” refers to a target particle for the first measurement sample, and the “target particle B” refers to a target particle for the third measurement sample. The CPU 301 stores the number acquired in step S307 in the SSD 304. In step S308, the number of cell complexes containing an activated T cell per 10,000 particles in the first measurement sample is acquired by subtracting the number of background particles per 10,000 particles from the number of target particles A per 10,000 particles. The CPU 301 stores the number acquired in step S308 (hereinafter, also referred to as “number X”) in the SSD 304. In step S309, the number of cell complexes containing an activated T cell per 10,000 particles in the third measurement sample (hereinafter, also referred to as “number Y”) is acquired by subtracting the number of background particles per 10,000 particles from the number of target particles B per 10,000 particles. The CPU 301 stores the number acquired in step S308 in the SSD 304. In step S310, the CPU 301 acquires a value obtained by subtracting the number X from the number Y, and the CPU 301 stores the value in the SSD 304.

In step S311, the CPU 301 compares the number acquired in step S310 with the predetermined threshold value stored in the SSD 304. When the acquired number is greater than or equal to the predetermined threshold value, the process proceeds to step S312. In step S312, the CPU 301 stores a determination result that the biological sample has been collected from a subject who is in a state of immune tolerance to a predetermined antigen in the SSD 304. In step S311, when the acquired number is less than the predetermined threshold value, the process proceeds to step S313. In step S313, the CPU 301 stores a determination result that the biological sample has been collected from a subject who is not in a state of immune tolerance to a predetermined antigen in the SSD 304. The CPU 301 may display the determination result of step S312 or S313 on the display unit 320.

### <Embodiment 2 of Cell Analyzer>

In the analysis processing of Embodiment 1 of the cell analyzer, the analysis was performed based on the captured image data, but the number of cell complexes containing an activated T cell may be analyzed from measurement data other than the image data. As a modification of the imaging unit 203, for example, referring to FIG. 11, the imaging unit 203 may be a detection unit that includes a light source 203a, a flow cell 203c, condenser lenses 203d, 203e, and 203j, a dichroic mirror 203k, fluorescent light receiving units 203l and 203n, and a forward scattered light receiving unit 203m, and the imaging unit 203 detects an optical signal.

The light source 203a is a semiconductor laser light source that can emit laser beams with a plurality of different wavelengths. In FIG. 11, the light irradiated from the light source 203a is laser beams of wavelengths λ1 and λ2. In Embodiment 2, the wavelength λ1 is, for example, about 645 nm, and the wavelength λ2 is, for example, about 490 nm. The condenser lens 203d collects the laser beam irradiated from the light source 203a. The condenser lens 203d guides the collected light to the measurement sample in the flow cell 203c. The laser beam irradiated from the light source 203a is irradiated onto the individual particle passing through the inside of the flow cell 203c. For example, when a T cell labeled with MTDR and FITC-labeled anti-CD4 antibody is irradiated with a laser beam, fluorescence with a wavelength λ3 (for example, about 665 nm) is generated from MTDR, and fluorescence with a wavelength λ4 (for example, about 515 nm) is generated from FITC. In addition, forward scattered light (FSC) is also generated from a particle irradiated with a laser beam. The condenser lens 203e collects the forward scattered light. The forward scattered light is received by the forward scattered light receiver 203m. The condenser lens 203j collects the fluorescence of wavelengths λ3 and λ4. The fluorescent light of wavelength λ3 is transmitted through the dichroihc mirror 203k to be received by the fluorescent light receiving unit 203l. The fluorescent light of wavelength λ4 is reflected by the dichroihc mirror 203k to be received by the fluorescent light receiving unit 203n. As the fluorescent light receiving unit and the forward scattering light receiving unit, for example, an avalanche photodiode, a photodiode or a photomultiplier tube can be used.

In this example, the optical signals obtained in step S22 in FIG. 6 are a forward scattered light signal and a fluorescence signal. In step S14 of FIG. 6, forward scattered light intensity, the fluorescence intensity of the fluorescence signal from MTDR (hereinafter, also referred to as “fluorescence intensity 1”), and the fluorescence intensity of the fluorescence signal from FITC (CD4) (hereinafter, also referred to as “fluorescence intensity 2”) are obtained as the parameters for analysis.

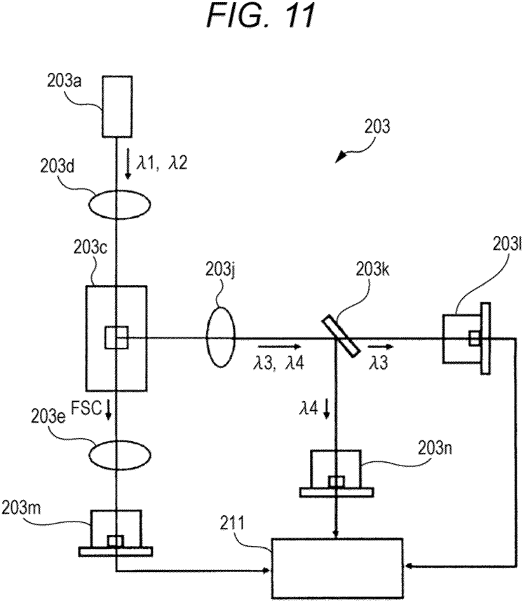

The flow of the analysis processing in step S15 of FIG. 6 will be described with reference to FIG. 12. However, the present invention is not limited to this analysis processing. In step S401, the CPU 301 randomly extracts measurement data of tens of thousands of particles (for example, 50,000 particles) from the measurement data of each of the first measurement sample and the second measurement sample acquired in step S13 of FIG. 6. In step S402, the CPU 301 extracts measurement data of a particle in which the fluorescence intensity 2 is greater than or equal to a predetermined threshold value from the measurement data extracted in step S101 for each measurement sample. Thereby, measurement data of a CD4-positive T cell or a particle containing the cell is extracted. In step S403, the CPU 301 extracts measurement data of a particle in which the forward scattered light intensity is greater than or equal to a predetermined threshold value from the measurement data extracted in step S402. The particle in which the forward scattered light intensity is greater than or equal to a predetermined threshold value may be a cell complex to which two to three cells adhere. In step S404, the CPU 301 extracts measurement data of a particle in which the fluorescence intensity of the fluorescence intensity 1 is greater than or equal to a predetermined threshold value from the measurement data extracted in step S403 for each measurement sample. The particle in which the fluorescence intensity of the fluorescence intensity 1 is greater than or equal to a predetermined threshold value may be an activated T cell or a particle containing the cell.

In step S405, the CPU 301 counts the particles extracted in step S404 for each measurement sample, the CPU 301 converts the number into the number per focused 10,000 particles, and the CPU 301 acquires the number of target particles per 10,000 particles and the number of background particles per 10,000 particles. The CPU 301 stores the number acquired in step S405 in the SSD 304. In step S406, the number of cell complexes containing an activated T cell per 10,000 particles in the first measurement sample is acquired by subtracting the number of background particles per 10,000 particles from the number of target particles per 10,000 particles. The CPU 301 may store the number acquired in step S406 in the SSD 304. The CPU 301 may display the number as an analysis result on the display unit 320. In addition, using the number acquired in step S406, determination of specific immune response to an antigen may be performed in the same manner as in steps S209 to S211 of FIG. 10A.

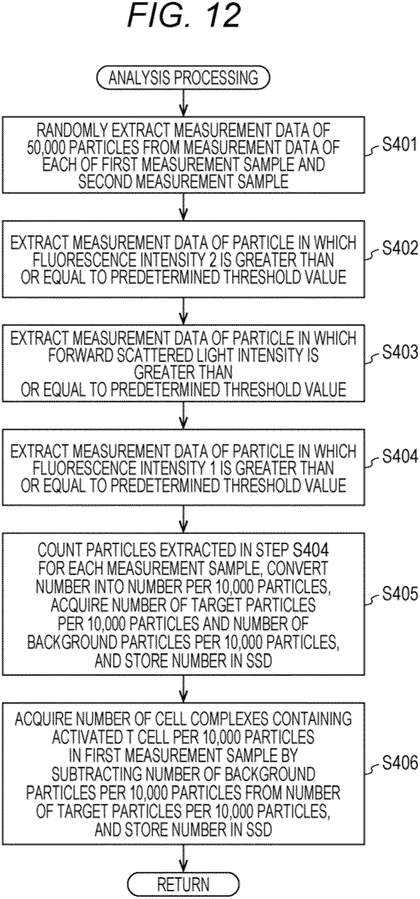

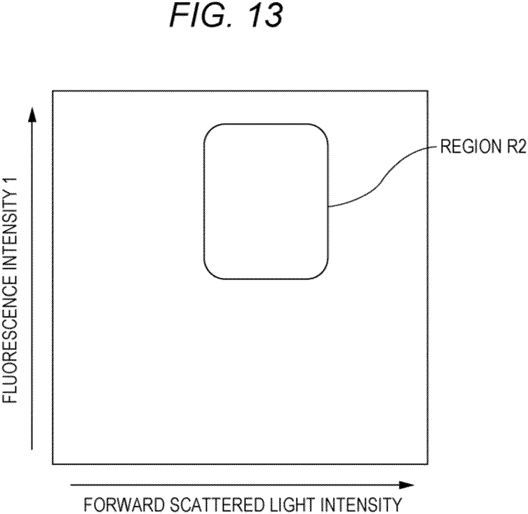

In step S404, the CPU 301 may create a scattergram in which the forward scattered light intensity is taken on X-axis and the fluorescence intensity 1 is taken on Y-axis. Referring to FIG. 13, region R2 is a region in which the forward scattered light intensity (X axis) is gated in a range of two to three cells, and the fluorescence intensity 1 (Y axis) is gated in a predetermined range reflecting the presence of activated mitochondria. For example, in the scattergram for the first measurement sample, a dot of the target particle may be displayed in the region R2. Also, in the scattergram for the second measurement sample, a dot of the background particle may be displayed in the region R2.

Hereinafter, the present invention will be described in more detail with reference to examples, but the present invention is not limited to these examples.

### EXAMPLES

### Example 1: Detection of Cell Complex of T Cell Activated by Antigen and B Cell

A measurement sample obtained by mixing a mouse spleen cell or a T cell line, a B cell line, and a predetermined peptide as an antigen was measured by IFC, and whether a cell complex containing an antigen-specific activated T cell could be selectively detected was examined.

### 1. Preparation of Biological Sample

A liquid containing a spleen cell or a T cell line of DO 11.10 mouse (DO11.10 T hybridoma cell line) (1 × 10⁶ cells/50 µL) modified to activate OVA was prepared using 10% FBS-containing RPMI-1640 medium (FUJIFILM Wako Pure Chemical Corporation). In addition, a liquid containing a B cell line (IIA1.6 B lymphoma cell line) (0.25 × 10⁶ cells/50 µL) as an antigen-presenting cell was prepared using 10% FBS-containing RPMI-1640 medium. The T cell line was genetically modified to express GFP-fused PD-1 protein by a conventional method. A staining buffer (50 µL) containing PE-Cy7-labeled anti-CD19 antibody (BioLegend, Inc.) was added in advance to the B cell line, and the mixture was incubated on ice for 20 minutes. This B cell line was washed once with the staining buffer, and then washed once with a warmed medium. An OVA peptide (OVA323-339 Eurofins) as an antigen was added to the washed B cell line, and pre-incubation was performed in a CO₂ incubator (37° C.) for 40 minutes to 90 minutes. The T cell line and the B cell line were obtained from Department of Immunology and Genomic Medicine, Graduate School of Medicine, Kyoto University.

### 2. Preparation of Measurement Sample and Labeling of T Cell Activation Marker

### Preparation of Measurement Sample

The liquid containing a spleen cell or a T cell line was mixed with the liquid containing a B cell line stimulated with an OVA peptide to prepare a measurement sample. The measurement sample was incubated in a CO₂ incubator (37° C.) for 10 minutes, 20 minutes, 40 minutes, 1 hour, 2 hours or 3 hours. For comparison, a measurement sample to which the antigen reagent was not added was also prepared. In addition, in order to confirm whether the cell complex was formed via TCR, a measurement sample was prepared by adding an anti-TCR antibody (Miltenyi Biotec) to the liquid containing a B cell line 10 minutes before the addition of the antigen.

### Fluorescence Staining of Mitochondria

Thirty minutes before the completion of the incubation of the measurement sample, a 30 to 100 nM MitoTracker (registered trademark) Deep Red (Invitrogen) solution was added in an amount of ⅑ of the reaction liquid amount. When the incubation time was shorter than 30 minutes, the stain solution was added in advance to the liquid containing a spleen cell or a T cell line. After completion of the incubation, the same amount of 4% PFA solution as the reaction liquid amount was added to the measurement sample, and the mixture was incubated at room temperature for 15 minutes. The measurement sample was centrifuged, and the cell was washed with the staining buffer. The cell was washed twice. In the case of a spleen cell, a staining buffer (50 µL) containing Mouse TruStain FcX (registered trademark) (BioLegend, Inc.) was added, and the mixture was incubated at room temperature for 5 minutes. After the incubation, a staining buffer (50 µL) containing a FITC-labeled anti-CD4 antibody (BD Bioscience) was added, and the mixture was incubated at room temperature for 15 minutes. The cell was then washed twice with the staining buffer.

### 3. Measurement and Data Analysis

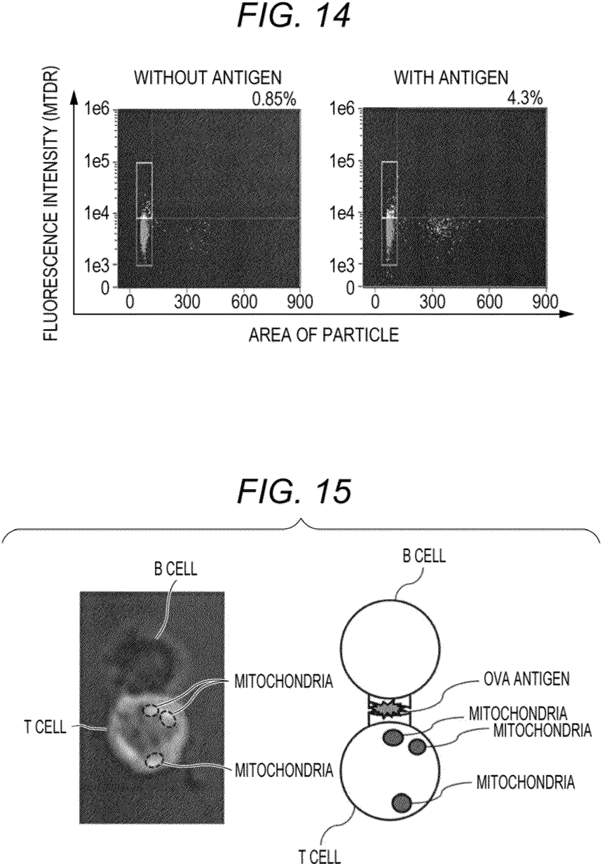

The measurement sample after staining was measured with an imaging flow cytometer MI-1000 (Sysmex Corporation). By the measurement, a bright field image and a fluorescence image were acquired for each particle in the measurement sample. Data of the acquired image was analyzed with IDEAS Application v6.0 (MERCK MILLIPORE), which is software for image analysis. First, images of 10,000 to 50,000 particles were acquired from the images acquired by MI-1000. Then, among the acquired images, the focused image was extracted by feature of the IDEAS, RMS 50-80. A particle in the extracted image was to be analyzed. The bright field image of the particle to be analyzed was analyzed with the feature of the IDEAS, Area, to acquire the area value of the particle in each image. In addition, the fluorescence image of the particle to be analyzed was analyzed with the intensity feature of the IDEAS, and the fluorescence intensity of the fluorescence signal generated from each of FITC and MitoTracker (registered trademark) Deep Red was acquired. An image of a particle with a fluorescence intensity derived from FITC higher than a predetermined threshold value was extracted. The particle in the image corresponded to a CD4-positive cell, i.e. a T cell. Next, a scattergram in which the area value of the particle was taken on X-axis and the fluorescence intensity was taken on Y-axis was created. As an example, a scattergram based on the area value of the particle and the fluorescence intensity of mitochondria of CD4-positive cell is shown in FIG. 14. In the scattergram, a particle having an area value corresponding to two to three cells and the fluorescence intensity derived from MitoTracker (registered trademark) Deep Red higher than a predetermined threshold value was selected. Data for each selected particle were extracted as data for a cell complex in which a T cell and a B cell adhere to each other, including an antigen-specific activated T cell. The number of the particles was counted as the number of cell complexes. In addition, an image of each selected particle was visually confirmed, and an image in which a cell complex of a T cell fluorescently stained with activated mitochondria and a B cell was actually imaged was extracted.

### 4. Results

As shown in FIG. 14, in the case where the antigen reagent was not added, the proportion of particles in a region where both the area value and the fluorescence intensity were greater than or equal to the predetermined threshold value on the scattergram was 0.85%. On the other hand, in the case where the antigen reagent was added, the proportion of the particles increased to 4.3%. FIG. 15 shows an example of an image of a particle in a region where both the area value of the particle and the fluorescence intensity were greater than or equal to the predetermined threshold value in the case where the antigen reagent was added, and a schematic diagram thereof. In the particle in the fluorescence image of FIG. 15, two cells adhered to form a cell complex. Since a FITC-derived fluorescence signal was observed on the surface of one cell and no fluorescence signal was particularly observed on the surface of the other cell, this cell complex was a cell complex of a CD4-positive T cell and a B cell. As indicated by a region surrounded by a broken line in the fluorescence image of FIG. 15, activated mitochondria were observed in the T cell of the cell complex. FIGS. 14 and 15 show results when a mouse spleen cell was used.

The frequency of particles having an area value corresponding to two to three cells with respect to the particle to be analyzed was compared between the case where the antigen reagent was added and the case where the antigen reagent was not added. The results are shown in FIG. 16. In addition, in the case where the antigen reagent was added (reaction time was 2 hours), the frequency of particles having an area value corresponding to two to three cells when the anti-TCR antibody was further added was examined. The results are shown in FIG. 17. As shown in FIG. 16, by the addition of antigen, the frequency of particles having an area value corresponding to two to three cells clearly increased. As shown in FIG. 17, the addition of the anti-TCR antibody significantly reduced the frequency of particles having an area value corresponding to two to three cells. From these, it was suggested that by the addition of antigen, a T cell and a B cell adhere to each other to form a cell complex, and the formation of this cell complex is mediated by TCR. FIGS. 16 and 17 show results when a mouse T cell line was used.

### Example 2: Detection of Cell Complex of T Cell Activated by Antigen and Antigen-Presenting Cell

A measurement sample obtained by mixing a human PBMC and various exogenous antigens different from the antigen of Example 1 was measured by IFC, and whether a cell complex containing an antigen-specific activated T cell could be selectively detected was examined.

### 1. Biological Samples and Antigens

As biological samples, PBMCs having high reactivity to a predetermined antigen were purchased from Cellular Technology Limited (hereinafter referred to as C. T. L.) and used. Specifically, PBMC reactive to a resident viral antigen, PBMC reactive to a protein antigen of Mycobacterium tuberculosis, and PBMC reactive to a mite antigen were used. The PBMCs were prepared to 1 × 10⁶ cells/100 µL using 10% FBS-containing RPMI-1640 medium (FUJIFILM Wako Pure Chemical Corporation) or CTL-Test medium (C. T. L.). Tuberculin PPD (JVaccines), which is a protein antigen of Mycobacterium tuberculosis, was used as a Mycobacterium tuberculosis antigen. CPI Positive control (C. T. L.) was used as a protein antigen of resident virus. XPB81D3A2.5 and XPB82D3A2.5 (Greer laboratories Inc.) were used as mite antigens. Staphylococcus aureus-derived Enterotoxin B (Sigma-Aldrich), which is a superantigen, was used as a positive control.

### 2. Preparation of Measurement Sample and Labeling of T Cell Activation Marker

### Preparation of Measurement Sample

The antigen reagent was prepared by diluting to an appropriate concentration with CTL-Test medium (C. T. L.) according to an attached document of each antigen. PBMC (1 × 10⁶ cells/100 µL) and the antigen reagent (100 µL) were mixed to prepare a measurement sample. For comparison, a measurement sample to which the antigen reagent was not added was also prepared. The measurement sample was incubated in a CO₂ incubator (37° C.) for 2 hours.

### Fluorescence Staining of pERK

After completion of the incubation of the measurement sample, a 4% PFA solution (200 µL) was added to the measurement sample, and the mixture was incubated at room temperature for 5 minutes. Thereafter, the measurement sample was placed on ice, and the mixture was incubated for 15 minutes. 100% Methanol cooled at 4° C. in advance was added to the measurement sample, and the mixture was incubated for about 10 minutes. The measurement sample was centrifuged, and the cell was washed with the staining buffer. The cell was washed twice. A staining buffer (50 µL) containing Human TruStain FcX (registered trademark) (BioLegend, Inc.) was added to the cell, and the mixture was incubated at room temperature for 5 minutes. After the incubation, a staining buffer (50 µL) containing an Alexa488 labeled anti-pERK antibody (Cell Signaling Technology) and an APC-labeled anti-CD4 antibody (Miltenyi Biotec) was added, and the mixture was incubated at 4° C. for 1 hour. The cell was then washed twice with the staining buffer.

### 3. Measurement and Data Analysis

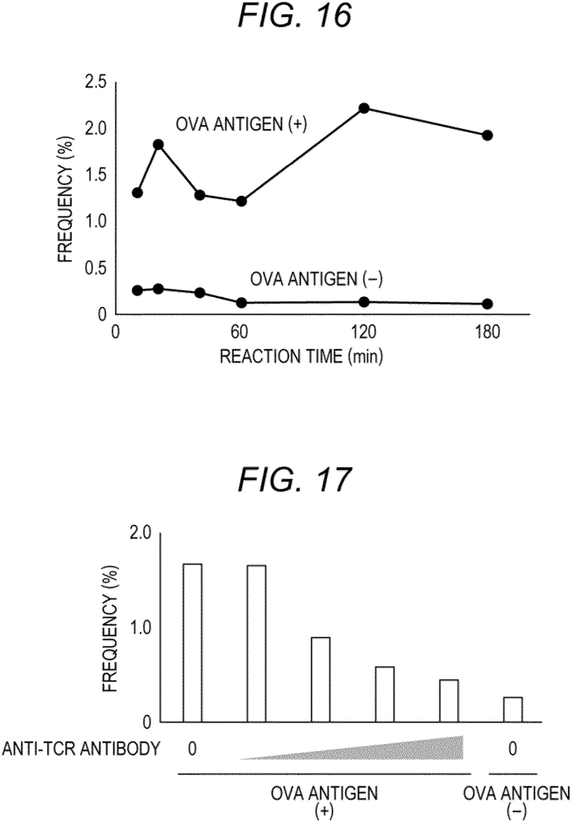

Measurement and data analysis were performed for each measurement sample in the same manner as in Example 1, and an image of a cell complex containing an antigen-specific activated T cell was acquired. Based on the area value of the particle and the value of the fluorescence intensity, a scattergram in which the area value of the particle was taken on X-axis and the fluorescence intensity in the CD4-positive cell was taken on Y-axis was created. In the scattergram, an image of a particle in a region where each of the area value and the fluorescence intensity was higher than a predetermined value was visually confirmed. Thereby, an image in which a cell complex of a CD4-positive T cell and an antigen-presenting cell was actually imaged was extracted. As an example, a scattergram of a measurement sample containing PBMC reactive to a viral antigen is shown in FIG. 18. In the figure, “Area-H pERK-H” refers to a region in which the area value corresponds to two to three cells and the fluorescence intensity derived from Alexa488 (pERK) is greater than or equal to the predetermined threshold value. “Area-H pERK-L” refers to a region in which the area value corresponds to two to three cells and the fluorescence intensity derived from Alexa488 (pERK) is lower than the predetermined threshold value. “Area-L pERK-H” refers to a region in which the area value is smaller than that of 2 cells and the fluorescence intensity derived from Alexa488 (pERK) is higher than the predetermined threshold value. In FIG. 18, the particle corresponding to the image in which the cell complex was visually confirmed was indicated by a symbol “+”. Of the particles indicated by “+”, an increase in pERK was observed in the particle in the Area-H pERK-H region, but pERK was hardly stained in the particle in the Area-H pERK-L region.

### 4. Results

As shown in FIG. 18, in the case where the antigen reagent was added, the number of particles in the Area-H pERK-H region increased as compared with the case where the antigen reagent was not added. In fact, the number of visually confirmed cell complexes (particles indicated by “+” in FIG. 18) also increased in the measurement sample to which the antigen reagent was added. Of the particles indicated by “+” in FIG. 18, strong staining of pERK was observed in the particle in the Area-H pERK-H region, but pERK was hardly stained in the particle in the Area-H pERK-L region. Therefore, it was shown that the addition of the antigen reagent increased the cell complex containing an activated T cell. Also in the scattergram of the measurement sample containing other PBMC, the distribution of the particles tended to be similar to that in FIG. 18. That is, in the case where the antigen reagent was added, the number of particles in the Area-H pERK-H region increased.

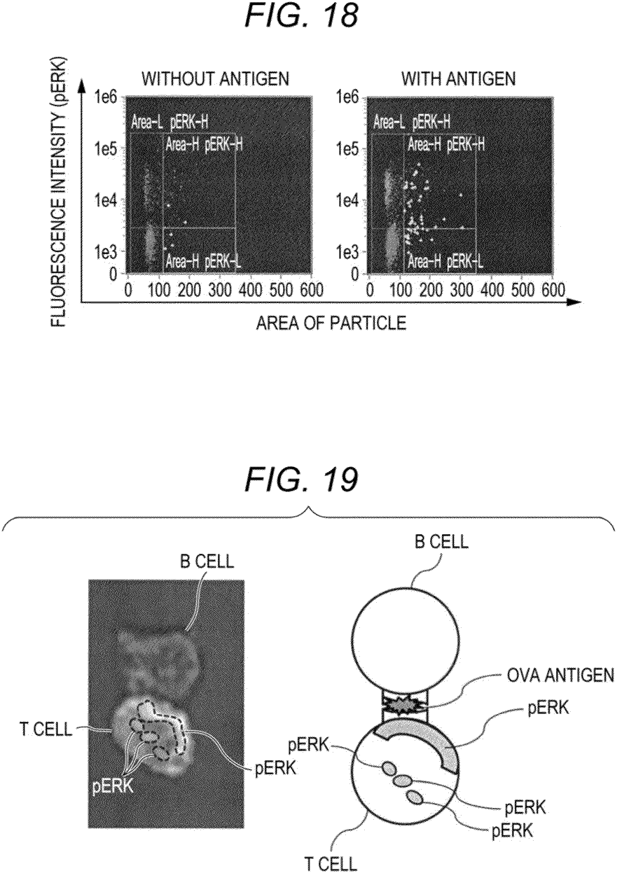

FIG. 19 shows an example of an image of a particle in a region where both the area value of the particle and the fluorescence intensity were greater than or equal to the predetermined threshold value in the case where the antigen reagent was added, and a schematic diagram thereof. In the fluorescence image of FIG. 19, a cell complex of a CD4-positive T cell in which a fluorescence signal derived from APC was observed on the cell surface and a B cell was observed. As indicated by a region surrounded by a broken line in the fluorescence image of FIG. 19, an increase in pERK was observed in the vicinity and central part of the site of adhesion to the B cell in the T cell of the cell complex. From these, it was shown that a cell complex containing an antigen-specific activated T cell can be detected by the information on the particle size and the information on mitochondrial membrane potential and pERK which are markers indicating activation of the T cell.

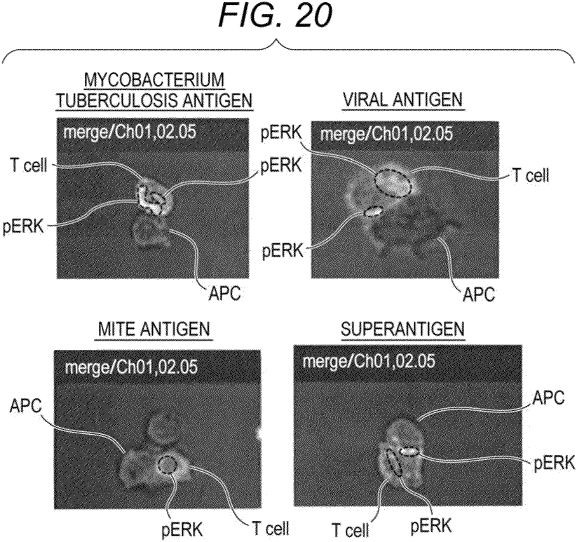

For each measurement sample, an example of an image of a cell complex in the case where the antigen reagent was added is shown in FIG. 20. As shown in FIG. 20, a cell complex of a CD4-positive T cell and an antigen-presenting cell was observed when either antigen was added. As indicated by a region surrounded by a broken line in FIG. 20, an increase in the amount of pERK was observed in the vicinity and central part of the sites of adhesion to the antigen-presenting cells in the T cells of these cell complexes. Therefore, it was shown that regardless of the type of antigen, a cell complex containing an antigen-specific activated T cell can be detected by information on the particle size and information on the activation of the T cell.

### Example 3: Examination of Detection Performance of T Cell Activated by Exogenous Antigen

The performance of the method for detecting a cell complex containing an activated T cell performed in Examples 1 and 2 was examined by comparison with an enzyme-linked immunospot (ELISpot) assay. The ELISpot assay is a method for measuring the number of T cells by adsorbing a cytokine secreted from an antigen-specific activated T cell to a solid phase and detecting it as a spot.

### 1. Biological Samples and Antigens

As biological samples, the various PBMCs used in Example 2, PBMC having low reactivity to a resident viral peptide antigen, PBMC having low reactivity to a protein antigen of Mycobacterium tuberculosis, and PBMC having low reactivity to a mite antigen were used. As antigens, in addition to the various antigens used in Example 2, CEFT-MHC Class II Control Peptide Pool “PLUS” (C. T. L.) was used as a peptide antigen of virus. ESAT-6 (Oxford Immunotec) which is a peptide antigen of Mycobacterium tuberculosis was used. The antigen reagent was prepared by diluting to an appropriate concentration with CTL-Test medium (C. T. L.) according to an attached document of each antigen.

### 2. Preparation and Measurement of Measurement Sample

### Preparation of Measurement Sample for IFC, Staining and Measurement Of Marker

A measurement sample to which the antigen reagent was added was prepared in the same manner as in Example 2. For comparison, a measurement sample to which the antigen reagent was not added was also prepared. For these measurement samples, fluorescence staining of mitochondria and CD4 was performed in the same manner as in Example 1. In fluorescence staining, Human TruStain FcX (registered trademark) (BioLegend, Inc.) was used in place of the Mouse TruStain FcX (registered trademark) (BioLegend, Inc.), and a FITC-labeled anti-CD4 antibody (Miltenyi Biotec, Inc.) was used in place of the FITC-labeled anti-CD4 antibody (BD Bioscience, Inc.). After staining, each measurement sample was measured with MI-1000 and data analysis was performed with IDEAS software in the same manner as in Example 1. Then, the cell complexes containing an antigen-specific activated T cell were counted. The acquired number of cell complexes was converted to the number per PBMC 1 × 10⁴ cells.

### Preparation and Measurement of Measurement Sample for ELISpot

The above antigen reagent (100 µL) was added to each well of a 96-well plate on which a capture antibody that specifically binds to IFNγ was immobilized. For comparison, a well to which a medium not containing the antigen was added was also prepared. The above PBMC (1.3 × 10⁵ to 4 × 10⁵ cells/100 µL) was added to the well, and the well was incubated in a CO₂ incubator (37° C.) for 24 hours. After washing each well to remove a cell, a detection antibody that specifically binds to IFNy was added, and the plate was incubated at 37° C. for 2 hours. After washing the well, an ALP-labeled secondary antibody was added, and the plate was incubated for 30 minutes. After washing the well, a substrate solution of ALP was added and each well spot was counted with an ELISpot reader.

### 3. Results

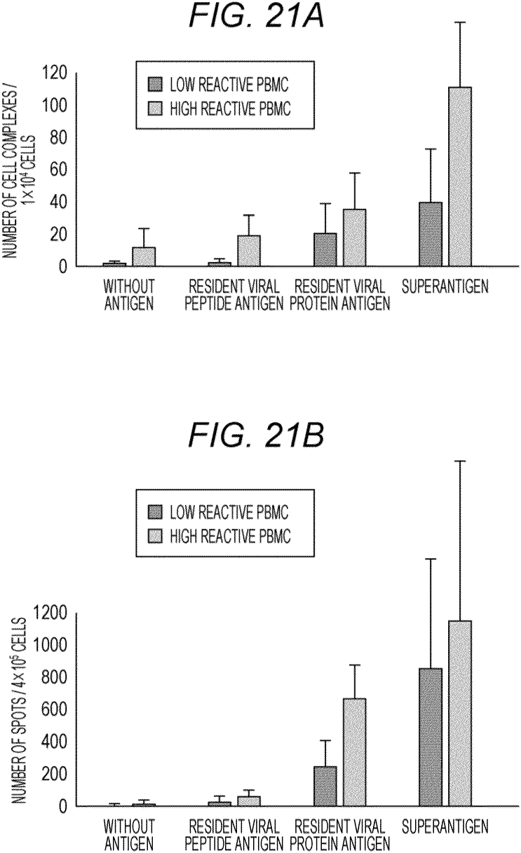

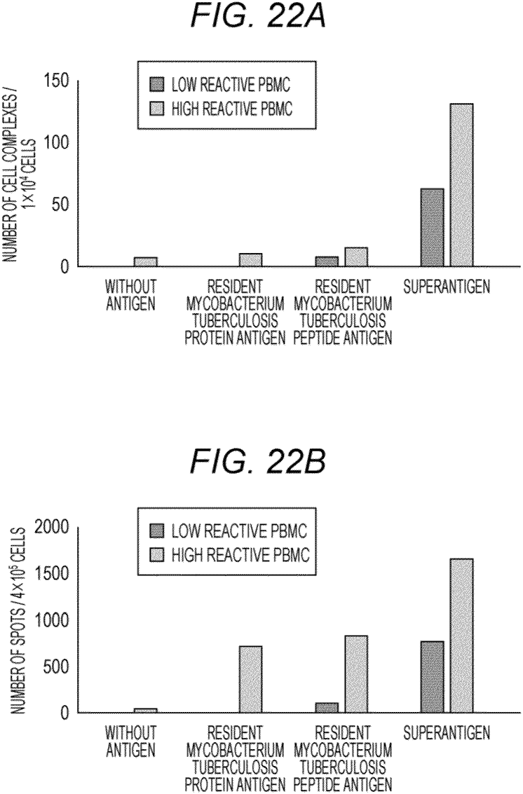

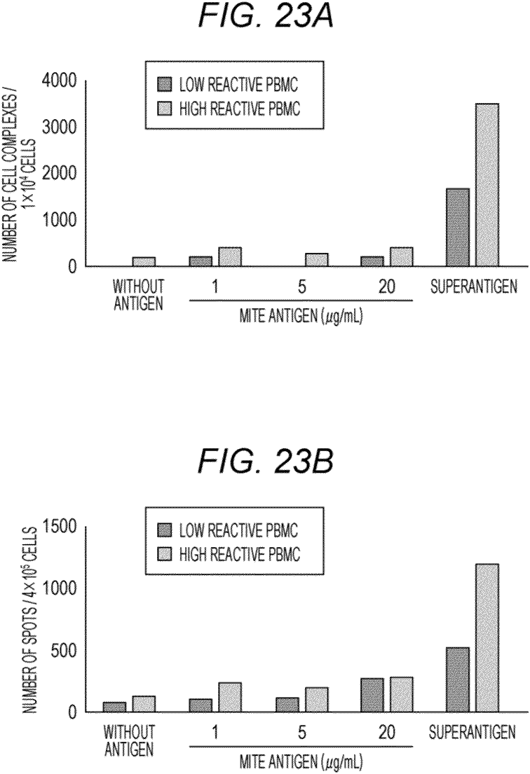

Graphs showing the number of cell complexes containing an activated T cell per PBMC 1 × 10⁴ cells in each measurement sample measured with MI-1000 are shown in FIG. 21A, FIG. 22A, and FIG. 23A. These complexes were detected based on the area value of the particle and the fluorescence intensity of mitochondria in the CD4-positive cell. In addition, graphs showing the number of spots converted per PBMC 4 × 10⁵ cells in each measurement sample detected by ELISpot method are shown in FIG. 21B, FIG. 22B, and FIG. 23B. As can be seen from FIG. 21A, FIG. 22A, and FIG. 23A, in either antigen, a large number of cell complexes containing an activated T cell were detected in PBMC having high reactivity to the antigen as compared with PBMC having low reactivity to the antigen. Thus, it was suggested that measurement by IFC can detect a number of cell complexes corresponding to reactivity of a T cell to the antigen. In addition, referring to FIG. 21B, FIG. 22B, and FIG. 23B, the measurement results by IFC correlated well with the results of the ELISpot method. According to the IFC, it was suggested that measurement results similar to those of the ELISpot method can be obtained in a short time.

### Example 4: Examination of Detection Performance of T Cell Activated by Autoantigen

Type I diabetes antigen IA-2 as an autoantigen was added to PBMC derived from a type I diabetic patient, and whether an activated T cell could be detected was examined. For comparison, measurement by the ELISpot method was also performed.

### 1. Biological Samples and Antigens

PBMC derived from a type I diabetic patient was purchased from Precision for Medicine and used as a biological sample. IA-2 protein (LifeSpan BioSciences Inc.) was used as an autoantigen. The same viral antigen as in Example 2 was used as a positive control. The antigen reagent was prepared by diluting to an appropriate concentration with CTL-Test medium (C.T.L.) according to an attached document of each antigen.

### 2. Preparation and Measurement of Measurement Sample

### Preparation of Measurement Sample for IFC, Staining and Measurement Of Marker

A measurement sample to which the antigen reagent was added was prepared in the same manner as in Example 2. For comparison, a measurement sample to which the antigen reagent was not added was also prepared. For these measurement samples, fluorescence staining of pERK and CD4 was performed in the same manner as in Example 2. After staining, each measurement sample was measured with MI-1000 and data analysis was performed with IDEAS software in the same manner as in Example 2. Then, the cell complexes containing an antigen-specific activated T cell were counted. The acquired number of cell complexes was converted to the number per PBMC 1 × 10⁴ cells.

### Preparation and Measurement of Measurement Sample for ELISpot

The above antigen reagent (100 µL) was added to each well of a 96-well plate on which a capture antibody that specifically binds to IFNγ was immobilized. For comparison, a well to which a medium not containing the antigen was added was also prepared. The cells (3 × 10⁵ cells/100 µL) were added to the well, and the well was incubated in a CO₂ incubator (37° C.) for 24 hours. After washing each well to remove a cell, a detection antibody that specifically binds to IFNy was added, and the plate was incubated at 37° C. for 2 hours. After washing the well, an ALP-labeled secondary antibody was added, and the plate was incubated for 30 minutes. After washing the well, a substrate solution of ALP was added and spots of each well were counted with an ELISpot reader.

### 3. Results

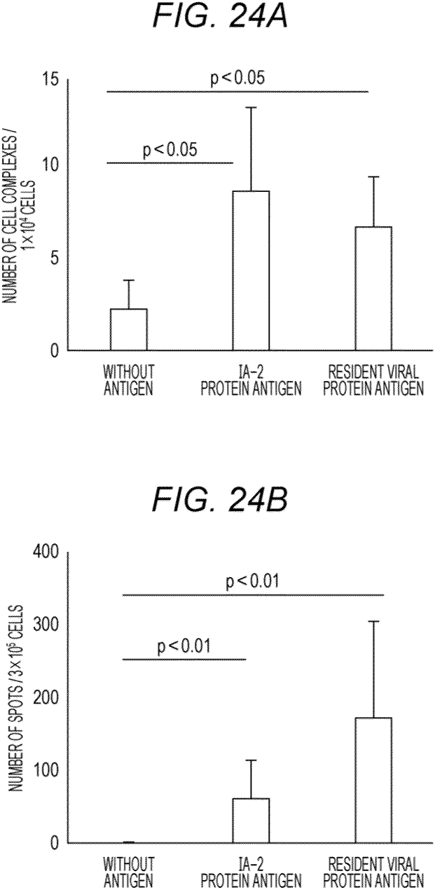

A graph showing the number of cell complexes containing an activated T cell per PBMC 1 × 10⁴ cells in each measurement sample measured with MI-1000 is shown in FIG. 24A. These complexes were detected based on the area value of the particle and the fluorescence intensity of pERK in the CD4-positive cell. In addition, a graph showing the number of spots per PBMC 3 × 10⁵ cells in each measurement sample detected by ELISpot method is shown in FIG. 24B. As can be seen in FIG. 24A, the measurement by IFC could detect a cell containing a T cell activated by IA-2 protein antigen. Referring to FIG. 24B, similarly in the ELISpot method, when the IA-2 protein antigen was added, the spot of IFNy released from the activated T cell increased. From these, it was suggested that the measurement by IFC can detect a T cell having reactivity to an autoantigen in a short time.

### Example 5: Involvement of MHC Molecule in Formation of Cell Complex of T Cell and Antigen-Presenting Cell in PBMC

In Example 1, it was suggested that the cell complex of a T cell line and a B cell line was formed by adhesion of a T cell line and a B cell line via TCR. The TCR is known to bind to an MHC molecule holding an antigen to form an immune synapse. In this example, whether a cell complex of a T cell and an antigen-presenting cell in PBMC to which an antigen was added was formed via MHC class II molecule of the antigen-presenting cell was examined. For comparison, measurement by the ELISpot method was also performed.

### 1. Biological Samples and Antigens

As a biological sample, PBMC reactive to viral protein antigen (C.T.L.) was used. The same viral protein antigen (CPI) as in Example 2 was used as an antigen. The antigen reagent was prepared by diluting the viral protein antigen with CTL-Test medium (C.T.L.) to an appropriate concentration.

### 2. Preparation and Measurement of Measurement Sample

### Preparation of Measurement Sample for IFC, Staining and Measurement Of Marker

A measurement sample to which the antigen reagent was added was prepared in the same manner as in Example 2. In addition, a measurement sample to which the antigen reagent and an anti-MHC-II antibody (Bio X Cell) or an IgG isotype control antibody (Bio X Cell) as a negative control antibody were added was prepared. The antibody was added to the biological sample 10 minutes prior to antigen addition. For comparison, a measurement sample to which the antigen reagent and the antibody were not added was also prepared. For these measurement samples, fluorescence staining of mitochondria and CD4 was performed in the same manner as in Example 3. After staining, each measurement sample was measured with MI-1000 in the same manner as in Example 3, and data analysis was performed with IDEAS software. Then, the cell complexes containing an antigen-specific activated T cell were counted. The acquired number of cell complexes was converted to the number per PBMC 1 × 10⁴ cells.

### Preparation and Measurement of Measurement Sample for ELISpot

The above antigen reagent (100 µL) was added to each well of a 96-well plate on which a capture antibody that specifically binds to IFNγ was immobilized. In addition, a well to which an antigen reagent containing an anti-MHC-II antibody or a negative control antibody was added was prepared. For comparison, a well to which a medium not containing the antigen was added was also prepared. The above PBMC (1.5 × 10⁵ cells/100 µL) was added to the well, and the well was incubated in a CO₂ incubator (37° C.) for 24 hours. After washing each well to remove a cell, a detection antibody that specifically binds to IFNy was added, and the plate was incubated at 37° C. for 2 hours. After washing the well, an ALP-labeled secondary antibody was added, and the plate was incubated for 30 minutes. After washing the well, a substrate solution of ALP was added and spots of each well were counted with an ELISpot reader. The acquired number of spots was converted to the number per PBMC 1.5 × 10⁵ cells.

### 3. Results

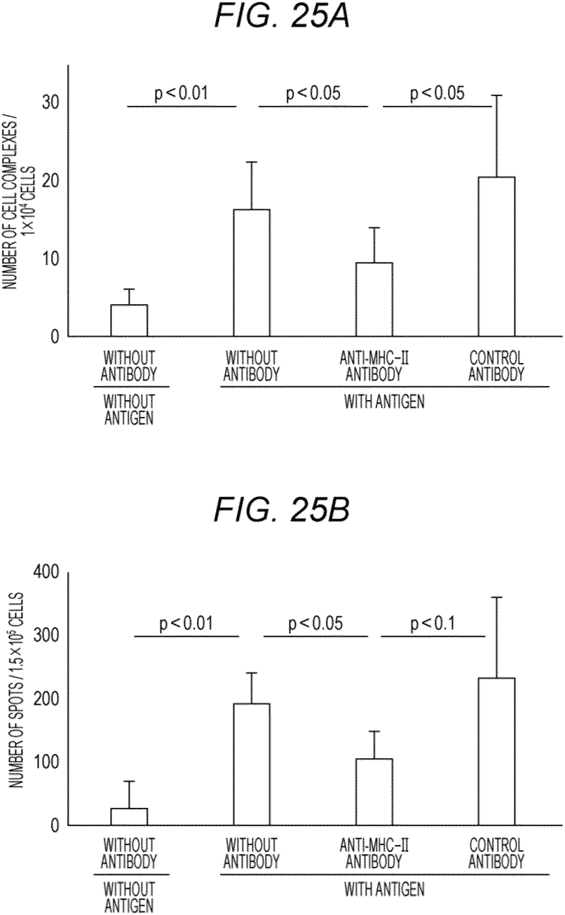

A graph showing the number of cell complexes containing an activated T cell per PBMC 1 × 10⁴ cells in each measurement sample measured with MI-1000 is shown in FIG. 25A. These complexes were detected based on the area value of the particle and the fluorescence intensity of mitochondria in the CD4-positive cell. In addition, a graph showing the number of spots per PBMC 1.5 × 10⁵ cells in each measurement sample detected by ELISpot method is shown in FIG. 25B. As can be seen from FIG. 25A, the number of cell complexes increased by the addition of antigen was significantly reduced by addition of anti-MHC-II antibody. On the other hand, when the IgG isotype control antibody as a negative control was added, no significant change was observed in the number of cell complexes. As shown in FIG. 25B, the ELISpot results were similar. From these, it was suggested that the cell complex of a T cell and an antigen-presenting cell in PBMC is formed by adhesion of a T cell and an antigen-presenting cell via MHC class II molecule. In view of the result of Example 1, it was suggested that formation of a cell complex of a T cell and an antigen-presenting cell was due to formation of an immune synapse of a TCR and an MHC molecule holding an antigen.

### Example 6: Discrimination of Immune Tolerance State Using Immune Tolerance Model System

When a measurement sample containing a T cell line and a B cell line expressing PD-L1 was measured by IFC, whether the cell complex was in a state of immune tolerance could be discriminated was examined.

### 1. Biological Samples and Antigens

As a biological sample, a liquid containing the T cell line used in Example 1 (DO11.10 T hybridoma cell line), a B cell line not expressing PD-L1 (IIA1.6 B lymphoma cell line) (hereinafter referred to as “PD-L1⁻”), a B cell line expressing PD-L1 at a low level (hereinafter referred to as “PD-L1^{low}”) or a B cell line expressing PD-L1 at a high level (hereinafter referred to as “PD-L1^{high}”) in a 10% FBS-containing RPMI-1640 medium (FUJIFILM Wako Pure Chemical Corporation) was prepared. The ratio of the number of T cells to the number of B cells in each of the solutions was 4 : 1. Each B cell line was obtained from Department of Immunology and Genomic Medicine, Graduate School of Medicine, Kyoto University. The same OVA peptide as in Example 1 was used as an antigen. The antigen reagent was prepared by diluting the OVA peptide with CTL-Test medium (C.T.L.) to an appropriate concentration.

### 2. Preparation and Measurement of Measurement Sample

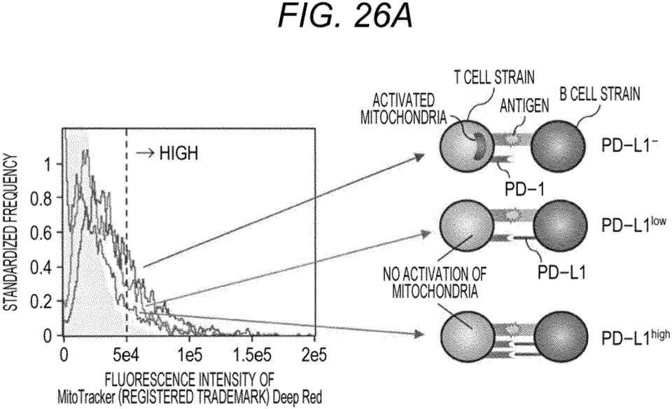

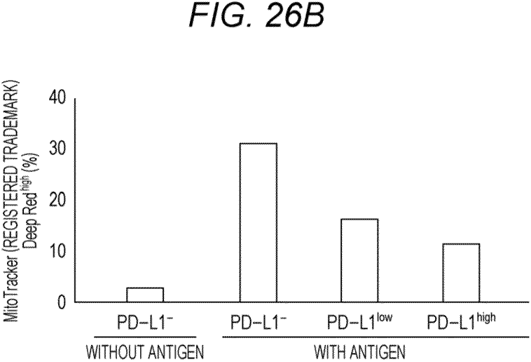

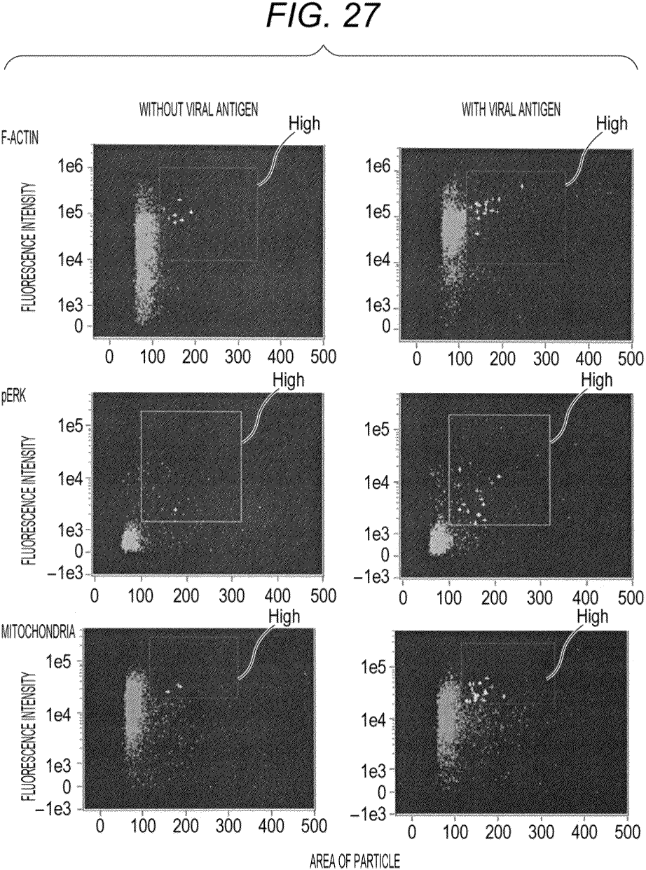

A measurement sample to which the antigen reagent was added was prepared in the same manner as in Example 1. The incubation time after addition of the antigen reagent was 2 hours. For these measurement samples, fluorescence staining of mitochondria and CD4 was performed in the same manner as in Example 1. After staining, each measurement sample was measured with MI-1000 and data analysis was performed with IDEAS software in the same manner as in Example 1. Then, the cell complexes were detected based on the area value of the particle, and the number of cell complexes according to the fluorescence intensity in the CD4-positive cell from MitoTracker (registered trademark) Deep Red was counted. The acquired number of cell complexes was converted to the number per PBMC 1 × 10⁴ cells. The results for each measurement sample are shown in FIG. 26A. In addition, a schematic diagram of a cell complex considered to be contained in each measurement sample is also shown.

The graph in FIG. 26A showed that the higher the PD-L1 expression level of the B cell line, the higher the number of cell complexes with a low fluorescence intensity of MitoTracker (registered trademark) Deep Red. This suggests that, as shown in the schematic diagram of the cell complex of FIG. 26A, PD-1 of the T cell and PD-L1 of the B cell are bound to each other, they are in a state of immune tolerance. In the graph of FIG. 26A, the number of cell complexes having a fluorescence intensity value greater than or equal to a predetermined fluorescence intensity value indicated by a dotted line was counted, and a ratio thereof was calculated. The results are shown in FIG. 26B. As can be seen from FIG. 26B, in the measurement sample containing PD-L1⁻, the cell complex containing a T cell having activated mitochondria increased by the addition of antigen, but in the measurement sample containing PD-L1^{low} or PD-L1^{hlgh.} such a cell complex decreased. From these, it was suggested that the measurement by IFC can discriminate whether the T cell in the cell complex is in a state of immune tolerance.

### Example 7: Study of Signal/Noise Ratio

The S/N ratio in the case of detecting the cell complex was examined based on the area value of the particle and the information on the activation of the T cell (activated mitochondria and pERK). For comparison, a cell complex based on the area value of the particle and information on F-actin that is a molecule constituting an immune synapse was also detected.

### 1. Biological Samples and Antigens

As a biological sample, human PBMC reactive to a resident viral antigen (C.T.L.) was used. The same viral antigen as in Example 2 was used as an antigen. An antigen reagent was prepared by diluting the viral antigen with CTL-Test medium (C.T.L.) to an appropriate concentration. A measurement sample to which the antigen reagent was added was prepared in the same manner as in Example 2. For comparison, a measurement sample to which the antigen reagent was not added was also prepared.

### 2. Labeling and Measurement of T Cell Activation Marker

### Fluorescence Staining and Measurement of Mitochondria or pERK

For each measurement sample, fluorescence staining of mitochondria or pERK and CD4 was performed in the same manner as in Example 2 or 3. Each measurement sample after staining was measured with MI-1000 in the same manner as in Example 2 or 3, and data analysis was performed with IDEAS software. Then, the cell complexes containing an antigen-specific activated T cell were counted. The acquired number of cell complexes was converted to the number per PBMC 1 × 10⁴ cells.

### Fluorescence Staining and Measurement of F-actin

Thirty minutes before the completion of the incubation of the measurement sample, a solution of 150 nM silicon rhodamine actin (SiR-Actin) (Cytoskeleton, Inc.) was added in an amount of ⅑ of the reaction liquid amount. SiR-Actin was a reagent that specifically stains F-actin. After completion of the incubation, the same amount of 4% PFA solution as the reaction liquid amount was added to the measurement sample, and the mixture was incubated at room temperature for 15 minutes. The measurement sample was centrifuged, and the cell was washed with the staining buffer. The cell was washed twice. A staining buffer (50 µL) containing Human TruStain FcX (registered trademark) (BioLegend, Inc.) was added to the cell, and the mixture was incubated at room temperature for 5 minutes. After the incubation, a staining buffer (50 µL) containing a FITC-labeled anti-CD4 antibody (Miltenyi Biotec) was added, and the mixture was incubated at room temperature for 15 minutes. The cell was then washed twice with the staining buffer.

Each measurement sample after staining was measured with MI-1000 and data analysis was performed with IDEAS software. First, images of 10,000 to 20,000 particles were acquired from the images acquired by MI-1000. Then, among the acquired images, a particle of the focus image was extracted by feature of the IDEAS, RMS 50-80, and was to be analyzed. The bright field image of the particle to be analyzed was analyzed with the feature of the IDEAS, Area, to acquire the area value of the particle in each image. In addition, the fluorescence image of the particle to be analyzed was analyzed with the feature of the IDEAS, Intensity, to acquire the fluorescence intensity of the fluorescence signal generated from FITC. Moreover, the fluorescence intensity of the fluorescence signal generated from SiR-Actin was acquired. In order to extract a CD4-positive cell, an image of a particle with a fluorescence intensity derived from FITC higher than a predetermined threshold value was extracted. Next, a scattergram in which the area value of the particle was taken on X-axis and the fluorescence intensity was taken on Y-axis was created. In the scattergram, a particle having an area value corresponding to two to three cells and the fluorescence intensity derived from SiR-Actin higher than a predetermined threshold value was selected. An image of each selected particle was visually confirmed, and an image in which a cell complex in which F-actin is localized on the adhesion surface between the T cell and the B cell was imaged was extracted. Localization of F-actin indicated formation of an immune synapse. The number of particles corresponding to the visually extracted image was counted as the number of cell complexes forming an immune synapse. The acquired number of cell complexes was converted to the number per PBMC 1 × 10⁴ cells.

### 3. Results

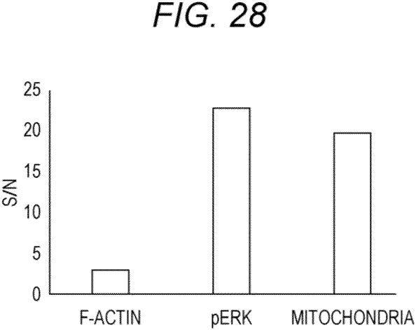

FIG. 27 shows an example of a scattergram in which the area value of the particle is taken on X-axis and the fluorescence intensity is taken on Y-axis for each measurement sample. In the figure, “MTDR” refers to MitoTracker (registered trademark) Deep Red. “High” refers to a region in which the area value corresponds to two to three cells and the fluorescence intensity derived from a fluorescent substance is greater than or equal to the predetermined threshold value. The symbol “+” in the High region of the upper panel (F-actin) indicates a particle corresponding to an image in which a cell complex forming an immune synapse is visually confirmed. The formation of an immune synapse was confirmed by the localization of F-actin on the adhesion surface between the T cell and the antigen-presenting cell. The symbol “+” in the High region of the middle panel (pERK) and the lower panel (mitochondria) indicates a particle corresponding to an image in which a cell complex is visually confirmed.

In each scattergram, the S/N ratio was calculated based on the number of counted particles. For the measurement sample in which mitochondria or pERK was fluorescently stained, the ratio of the number of cell complexes containing an activated T cell in the measurement sample to which an antigen was added to the number of cell complexes containing an activated T cell in the measurement sample to which an antigen was not added was calculated as the S/N ratio. For the measurement sample fluorescently stained with F-actin, the ratio of the number of cell complexes forming an immune synapse in the measurement sample to which an antigen was added to the number of cell complexes forming an immune synapse in the measurement sample to which an antigen was not added was calculated as the S/N ratio. The results are shown in FIG. 28.

Referring to FIG. 27, when any of F-actin, pERK and mitochondria was used as an index, the number of particles in the High region appeared to be increased in the measurement sample to which an antigen was added, as compared with the measurement sample to which an antigen was not added. On the other hand, as shown in FIG. 28, the S/N ratio was both 20 or more when pERK or mitochondria was used as an index, but S/N ratio was 5 or less when F-actin was used as an index. As described above, it was shown that a cell complex containing an activated T cell can be detected on a low background by using pERK or mitochondria which is a marker indicating activation of the T cell as an index, rather than using F-actin which is a marker indicating formation of an immune synapse as an index.

### Example 8: Detection of Release of Immune Tolerance by Addition of Nivolumab (1)

PBMC may include a T cell expressing PD-1 and an antigen-presenting cell expressing PD-L1. When such a T cell and an antigen-presenting cell form a cell complex, it is considered that the binding between the PD-1 and the PD-L1 causes the T cell in the cell complex to be in a state of immune tolerance. It is considered that when an immune checkpoint inhibitor nivolumab (anti-PD-1 antibody) is added thereto, immune tolerance is released, and the number of cell complexes containing an activated T cell further increases. In this example, whether it could be detected that the immune tolerance of T cell was released by the addition of nivolumab by measurement by IFC was examined.

### 1. Biological Samples and Antigens

The same human PBMC as in Example 7 was used as a biological sample. The same viral antigen as in Example 2 was used as an antigen. An antigen reagent was prepared by diluting the viral antigen with CTL-Test medium (C.T.L.) to an appropriate concentration.

### 2. Preparation and Measurement of Measurement Sample

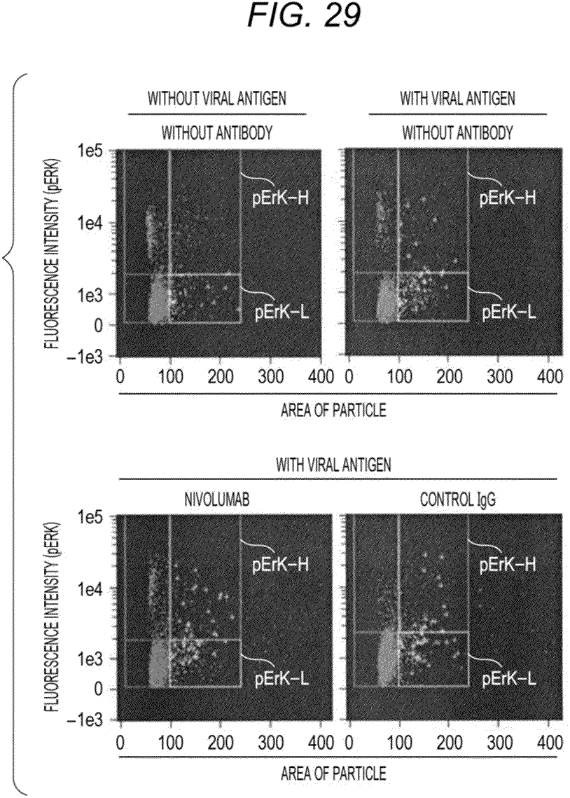

A measurement sample to which the antigen reagent was added was prepared in the same manner as in Example 2. In addition, a measurement sample to which the antigen reagent and nivolumab (absolute antibody) or control IgG (absolute antibody) were added was prepared. The antibody was added to the biological sample 10 minutes prior to antigen addition. For comparison, a measurement sample to which the antigen reagent and the antibody were not added was also prepared. For these measurement samples, fluorescence staining of pERK and CD4 was performed in the same manner as in Example 2. After staining, each measurement sample was measured with MI-1000 and data analysis was performed with IDEAS software in the same manner as in Example 2. The number of cell complexes containing an activated T cell obtained by analysis was converted to the number per PBMC 1 × 10⁴ cells. An example of a scattergram obtained by measuring each measurement sample by IFC is shown in FIG. 29. In the figure, “pErk-H” refers to a region in which the area value corresponds to two to three cells and the fluorescence intensity in the CD4-positive cell derived from Alexa488 is greater than or equal to the predetermined threshold value. “pErk-L” refers to a region in which the area value corresponds to two to three cells and the fluorescence intensity in the CD4-positive cell derived from Alexa488 is lower than the predetermined threshold value. In FIG. 29, the particle corresponding to the image in which the cell complex was visually confirmed was indicated by a symbol″+”.

In each scattergram, an image of each particle in the pErk-H region was visually confirmed, and an image in which a cell complex containing a T cell was actually imaged was extracted. The number of particles corresponding to the extracted image was counted as the number of cell complexes containing an antigen-specific activated T cell. For comparison, regardless of the value of the fluorescence intensity, particles having an area value corresponding to two to three cells were counted. These particles were composed of a cell complex containing a T cell activated by an antigen (particles in the pErk-H region) and a cell complex containing a T cell not activated (particles in the pErk-L region).

### 3. Results

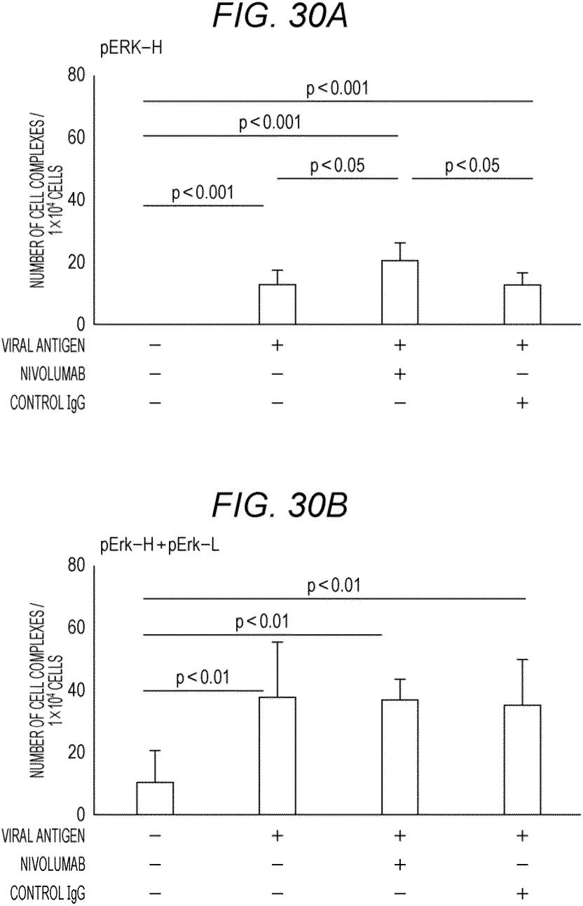

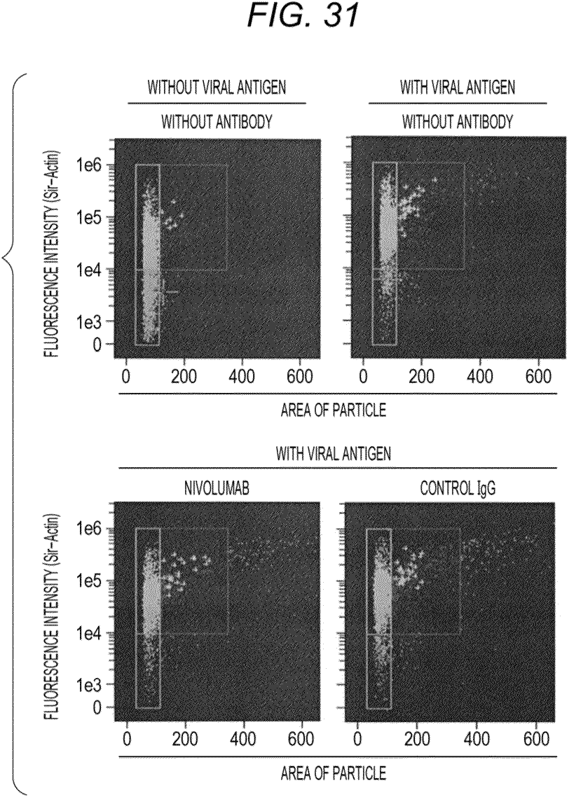

In FIG. 29, in any of the measurement samples, an increase in pERK was observed in the particle indicated by “+” in the High region. In the particle indicated by “+” in the Low region, pERK was hardly stained. A graph showing the number of cell complexes containing an activated T cell per PBMC 1 × 10⁴ cells in each measurement sample is shown in FIG. 30A. In addition, a graph showing the number of cell complexes per PBMC 1 × 10⁴ cells in each measurement sample is shown in FIG. 30B. Referring to FIG. 30A, the addition of viral antigen increased the number of cell complexes containing an activated T cell. By adding nivolumab with antigen, the number of cell complexes was significantly increased. On the other hand, when a control antibody was added together with the antigen, the number of cell complexes was significantly reduced as compared with the case where nivolumab was added, and was comparable to the case where only the antigen was added. The graph of FIG. 30B shows the total number of particles in the pErk-H region and particles in the pErk-L region, i.e., the number of cell complexes formed by the addition of antigen regardless of whether the T cell is activated. Referring to FIG. 30B, the number of cell complexes themselves hardly changed between the case where only the antigen was added and the case where the antigen and nivolumab or the control antibody were added. Therefore, the results shown in the graph of FIG. 30A indicate that the immune tolerance of the T cell whose activity was suppressed by the immune tolerance was released by nivolumab, and the number of cell complexes containing an activated T cell increased. From these, it was suggested that measurement by IFC can detect release of immune tolerance of T cell by an immune checkpoint inhibitor.

### Comparative Example 1

In this comparative example, it was examined whether the detection of release of immune tolerance performed in Example 8 can also be performed by the detection of immune synapse by IFC.

### 1. Preparation and Measurement of Measurement Sample

The biological sample and the antigen reagent were the same as those in Example 8. A measurement sample was prepared in the same manner as in Example 8. For each measurement sample, fluorescence staining of F-actin and CD4 was performed in the same manner as in Example 7. After staining, each measurement sample was measured with MI-1000 and data analysis was performed with IDEAS software in the same manner as in Example 7. An example of a scattergram obtained by measuring each measurement sample by IFC is shown in FIG. 31. In each scattergram, an image of a particle having an area value corresponding to two to three cells and the fluorescence intensity was higher than a predetermined threshold value were visually confirmed. In FIG. 31, the particle corresponding to the image in which the cell complex in which F-actin was localized on the adhesion surface between the T cell and the antigen-presenting cell was visually confirmed was indicated by a symbol “+”. The number of particles corresponding to the image in which the cell complex was confirmed was counted as the number of cell complexes in which an immune synapse was formed. In addition, the number of particles corresponding to the image in which the cell complex was confirmed was counted as the number of cell complexes by visual observation regardless of whether an immune synapse was formed. The acquired number of cell complexes was converted to the number per PBMC 1 × 10⁴ cells.

### 2. Results

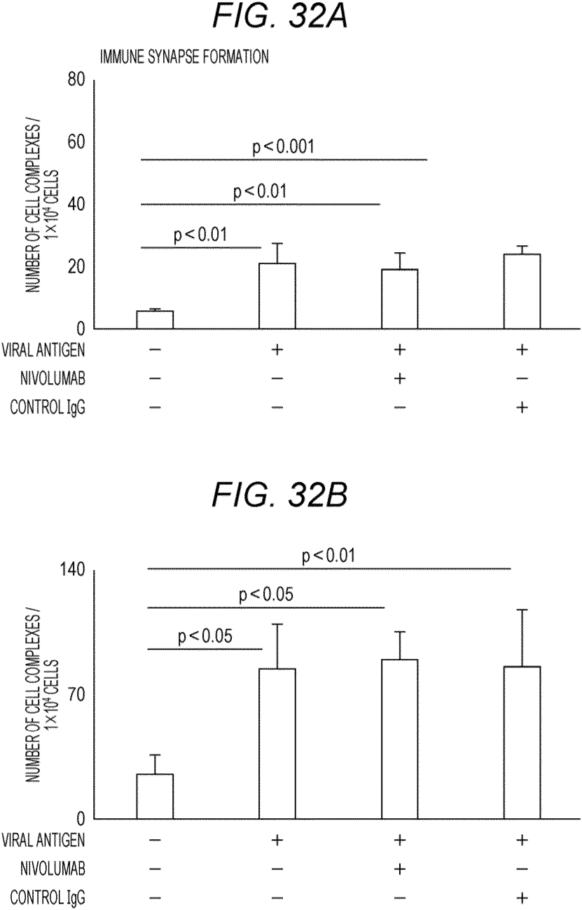

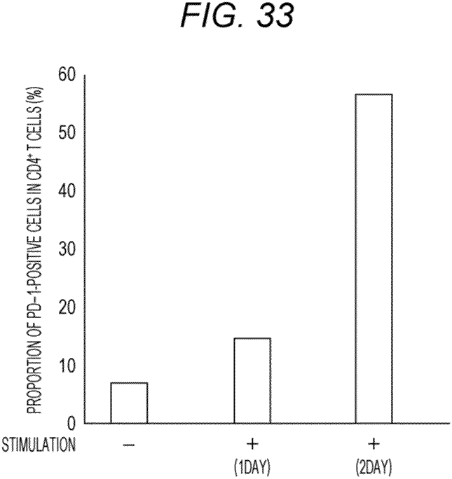

A graph showing the number of cell complexes in which an immune synapse was formed per PBMC 1 × 10⁴ cells in each measurement sample is shown in FIG. 32A. A graph showing the number of cell complexes per PBMC 1 × 10⁴ cells in each measurement sample is shown in FIG. 32B. Referring to FIG. 32A, the number of cell complexes themselves in which an immune synapse was formed hardly changed between the case where only the antigen was added and the case where the antigen and nivolumab or the control antibody were added. Referring to FIG. 32B, the number of cell complexes themselves was similar. From these, it was shown that it is difficult to detect release of immune tolerance of T cell by an immune checkpoint inhibitor by detecting formation of an immune synapse by IFC.

### Example 9: Detection of Release of Immune Tolerance by Addition of Nivolumab (2)

PD-1 is known as a molecule that expresses on the surface of activated T cell to negatively regulate an immune response by the T cell. In this example, the same examination as in Example 8 was performed using PBMC in which expression of PD-1 was promoted in the T cell by immune stimulation with CD3/CD28 beads as a biological sample.

### 1. Preparation of Biological Sample

CD3/CD28 beads (Gibco) were added to the same human PBMC as in Example 7, and the mixture was cultured for 1 day or 2 days. The CD3/CD28 beads are magnetic beads in which an anti-CD3 antibody and an anti-CD28 antibody are immobilized on the surface. For comparison, PBMC without CD3/CD28 beads was also cultured. After the culturing, the cells were fixed, washed, and blocked with Human TruStain FcX (registered trademark) (BioLegend, Inc.) in the same manner as in Example 1. Then, the cells were fluorescently stained using a PE-labeled anti-PD-1 antibody (BioLegend, Inc.) and a FITC-labeled anti-CD4 antibody (Miltenyi Biotec, Inc.). The fluorescently stained cells were measured with an imaging flow cytometer MI-1000 (Sysmex Corporation), and the number of CD4-positive T cells and the ratio of PD-1-positive cells in the T cells were calculated. The results are shown in FIG. 33. As shown in FIG. 33, by stimulating with CD3/CD28 beads for 1 day, the number of T cells expressing PD-1 moderately increased as compared with the case of not stimulating. From this result, it was decided to use PBMC stimulated with CD3/CD28 beads for 1 day as a biological sample.

### 2. Preparation of Antigen Reagent and Measurement Sample

The same viral antigen as in Example 2 was used as an antigen. An antigen reagent was prepared by diluting the viral antigen with CTL-Test medium (C.T.L.) to an appropriate concentration. A measurement sample to which the antigen reagent was added was prepared in the same manner as in Example 2. For comparison, a measurement sample to which the antigen reagent was not added was also prepared. A measurement sample was prepared in the same manner as in Example 8. For each measurement sample, fluorescence staining of mitochondria and CD4 was performed in the same manner as in Example 3.

### 3. Measurement of Measurement Sample

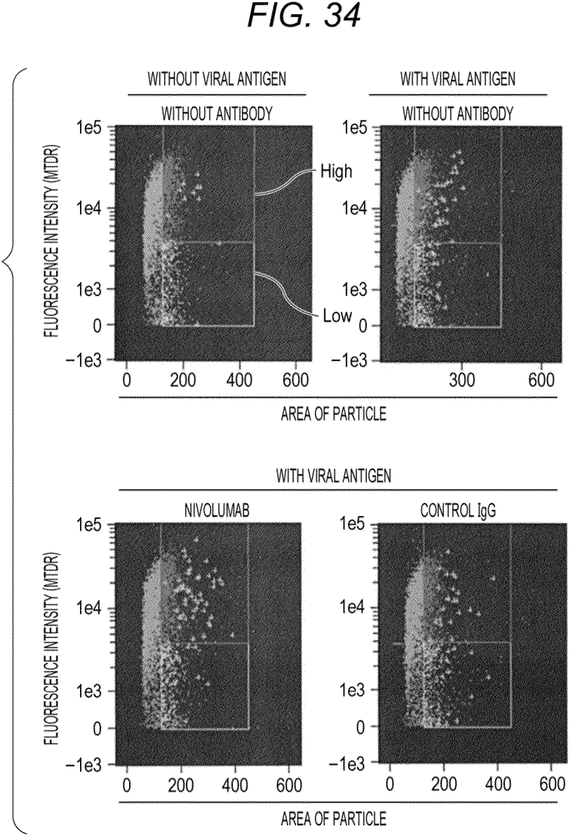

Each measurement sample was measured with MI-1000 and data analysis was performed with IDEAS software in the same manner as in Example 2. The number of cell complexes containing an activated T cell obtained by analysis was converted to the number per PBMC 1 × 10⁴ cells. An example of a scattergram obtained by measuring each measurement sample by IFC is shown in FIG. 34. In the figure, “MTDR” refers to MitoTracker (registered trademark) Deep Red. “High” refers to a region in which the area value corresponds to two to three cells and the fluorescence intensity in a MTDR-derived CD4-positive cell is greater than or equal to the predetermined threshold value. “Low” refers to a region in which the area value corresponds to two to three cells and the fluorescence intensity in a MTDR-derived CD4-positive cell is lower than the predetermined threshold value. In FIG. 34, the particle corresponding to the image in which the cell complex was visually confirmed was indicated by a symbol “+”.

### 4. Results

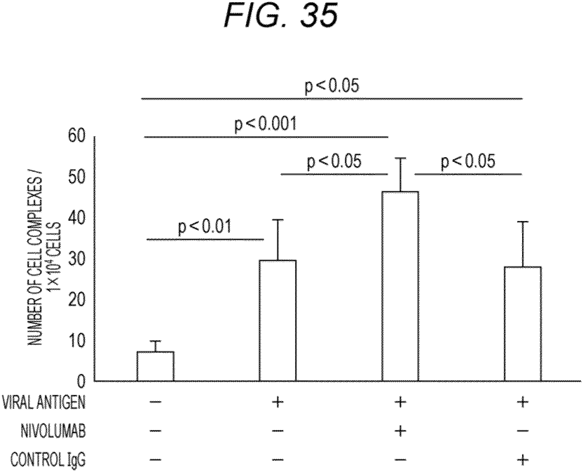

In FIG. 34, in any of the measurement samples, activated mitochondria were observed in the particle indicated by “+” in the High region. In the particle indicated by “+” in the Low region, mitochondria were hardly stained. A graph showing the number of cell complexes containing an activated T cell per PBMC 1 × 10⁴ cells in each measurement sample is shown in FIG. 35. Referring to FIG. 35, the addition of viral antigen increased the number of cell complexes containing an activated T cell. By adding nivolumab to the biological sample before the addition of antigen, the number of cell complexes was significantly increased. On the other hand, when a control antibody was added to the biological sample before the addition of antigen, the number of cell complexes was significantly reduced as compared with the case where nivolumab was added, and was comparable to the case where only the antigen was added. This result indicates that the immune tolerance of the T cell whose activity was suppressed by immune tolerance was released by nivolumab, and the number of cell complexes containing an activated T cell increased. From these, it was suggested that measurement by IFC can detect release of immune tolerance of T cell by an immune checkpoint inhibitor.

### Example 10: Detection of Cell Complex Containing Activated T Cell by Algorithm Analysis of Measurement Data

In this example, a cell complex containing an activated T cell in a measurement sample was detected only by analysis of measurement data according to an algorithm without visual confirmation of an image. This detection result was compared with the result of detecting a cell complex containing an activated T cell by visual confirmation of an image.

### 1. Biological Samples and Antigens

The same human PBMC as in Example 7 was used as a biological sample. The same viral antigen as in Example 2 was used as an antigen. An antigen reagent was prepared by diluting the viral antigen with CTL-Test medium (C.T.L.) to an appropriate concentration.

### 2. Preparation and Measurement of Measurement Sample

A measurement sample to which the antigen reagent was added was prepared in the same manner as in Example 2. For comparison, a measurement sample to which the antigen reagent was not added was also prepared. For each measurement sample, fluorescence staining of pERK and CD4 was performed in the same manner as in Example 2. Each measurement sample after staining was measured with MI-1000 in the same manner as in Example 2.

### 3. Data Analysis

### Detection by Algorithm Analysis of Measurement Data

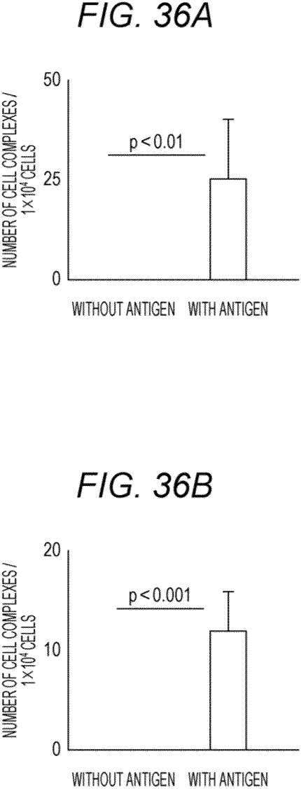

Data analysis was performed as follows. Images of 5,000 to 50,000 particles were acquired from the images acquired by MI-1000. Then, among the acquired images, the focused image was extracted by feature of the IDEAS, RMS 50-80. A particle in the extracted image was to be analyzed. The fluorescence image of the particle to be analyzed was analyzed with feature of the IDEAS. For each measurement sample, the fluorescence intensity of the fluorescence signal generated from each of APC (CD4) and Alexa488 (pERK) was acquired. An image of a particle with a fluorescence intensity derived from APC higher than a predetermined threshold value was extracted. The extracted bright field image of the particles was analyzed with the feature of the IDEAS, Area, to extract particles having an area value corresponding to two to three cells. From the extracted particles, particles having a fluorescence intensity in the CD4-positive cell derived from Alexa488 higher than the predetermined threshold value were extracted. From the extracted particles, particles having a fluorescence signal area value of APC corresponding to one cell were extracted. The number of extracted particles was counted as the number of cell complexes containing an activated T cell. The acquired number of cell complexes was converted to the number per PBMC 1 × 10⁴ cells. The number of cell complexes for the measurement sample not containing an antigen reagent was subtracted from the number of cell complexes for the measurement sample to which an antigen reagent was added. The obtained value was acquired as the number of cell complexes per PBMC 1 × 10⁴ cells in the measurement sample to which the antigen reagent was added. The results are shown in FIG. 36A.

### Detection by Visual Confirmation of Image

Data analysis was performed with IDEAS software in the same manner as in Example 1. The extracted image of the cell complex containing an activated T cell was visually confirmed, the image in which the cell complex containing a T cell fluorescently stained with pERK was actually imaged was extracted, and the number of cell complexes was counted. The acquired number of cell complexes was converted to the number per PBMC 1 × 10⁴ cells. The number of cell complexes for the measurement sample not containing an antigen reagent was subtracted from the number of cell complexes for the measurement sample to which an antigen reagent was added. The obtained value was acquired as the number of cell complexes per PBMC 1 × 10⁴ cells in the measurement sample to which the antigen reagent was added. The results are shown in FIG. 36B.

### 4. Results

As shown in FIG. 36A and FIG. 36B, the addition of viral antigen significantly increased the number of cell complexes containing an activated T cell. When FIG. 36A and FIG. 36B were compared with each other, the detection result only by the algorithm analysis of measurement data was similar to the detection result by the visual confirmation of an image. Therefore, it was shown that a cell complex containing an activated T cell can be detected even by detection only by the algorithm analysis of measurement data without the visual confirmation of an image.

### Example 11: Detection of Release of Immune Tolerance by Algorithm Analysis of Measurement Data

In this example, whether it could be detected that the immune tolerance of T cell was released by the addition of nivolumab only by the algorithm analysis of measurement data by an algorithm without visual confirmation of an image was examined. This detection result was compared with the detection result by visual confirmation of an image.

### 1. Biological Samples and Antigens

The same human PBMC as in Example 7 was used as a biological sample. The same viral antigen as in Example 2 was used as an antigen. An antigen reagent was prepared by diluting the viral antigen with CTL-Test medium (C.T.L.) to an appropriate concentration.

### 2. Preparation and Measurement of Measurement Sample

A measurement sample to which the antigen reagent was added was prepared in the same manner as in Example 2. A measurement sample to which the antigen reagent and nivolumab or control IgG were added was prepared in the same manner as in Example 8. For comparison, a measurement sample to which the antigen reagent and the antibody were not added was also prepared. For these measurement samples, fluorescence staining of pERK and CD4 was performed in the same manner as in Example 2. After staining, each measurement sample was measured with MI-1000 in the same manner as in Example 2.

### 3. Data Analysis

### Detection by Algorithm Analysis of Measurement Data

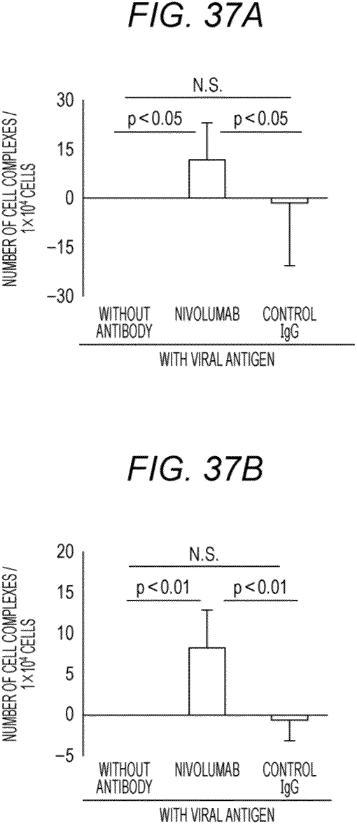

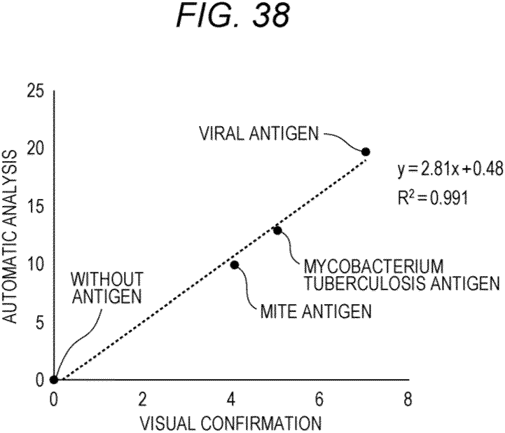

Algorithm analysis of measurement data was performed in the same manner as in Example 10, and the number of cell complexes containing an activated T cell per PBMC 1 × 10⁴ cells in each measurement sample was acquired. The number of cell complexes for the measurement sample not containing an antigen reagent was subtracted from the number of cell complexes for each measurement sample to which an antigen reagent was added. The obtained value was acquired as the number of cell complexes per PBMC 1 × 10⁴ cells in each measurement sample to which the antigen reagent was added. Next, the number of cell complexes for the measurement sample containing an antigen reagent and not containing an antibody was subtracted from the number of cell complexes for each measurement sample to which the antigen reagent and the antibody were added. The obtained value was acquired as the number of cell complexes per PBMC 1 × 10⁴ cells in the measurement sample to which the antigen reagent and the antibody were added. The results are shown in FIG. 37A.

### Detection by Visual Confirmation of Image

Data analysis was performed with IDEAS software in the same manner as in Example 2. The extracted image of the cell complex containing an activated T cell was visually confirmed, and the number of cell complexes of a T cell fluorescently stained with pERK and a B cell was counted in the same manner as in Example 10. The acquired number of cell complexes was converted into the number per PBMC 1 × 10⁴ cells to acquire the number of cell complexes containing an activated T cell per PBMC 1 × 10⁴ cells in each measurement sample. The number of cell complexes per PBMC 1 × 10⁴ cells in the measurement sample to which the antigen reagent and the antibody were added was acquired in the same manner as in the above (1). The results are shown in FIG. 37B.

### 4. Results

As shown in FIG. 37A and FIG. 37B, the addition of viral antigen and nivolumab significantly increased the number of cell complexes containing an activated T cell. On the other hand, in the measurement sample to which a viral antigen and control IgG were added, the number of cell complexes containing an activated T cell did not increase. This result indicates that the immune tolerance of the T cell whose activity was suppressed by immune tolerance was released by nivolumab, and the number of cell complexes containing an activated T cell increased. When FIG. 37A and FIG. 37B were compared with each other, the detection result only by the algorithm analysis of measurement data was similar to the detection result by the visual confirmation of an image. Therefore, it was shown that release of immune tolerance of T cell by an immune checkpoint inhibitor can be detected even by detection only by the algorithm analysis of measurement data without the visual confirmation of an image.

### Example 12: Correlation Between Analysis of Measurement Data by Algorithm and Analysis by Visual Confirmation

In this example, whether there is a correlation between the detection result by algorithm analysis of measurement data according to an algorithm and the detection result by visual confirmation of an image was examined with respect to the number of cell complexes containing an activated T cell.

### 1. Biological Samples and Antigens

The same human PBMC as in Example 7 was used as a biological sample. The same viral antigen, Mycobacterium tuberculosis antigen and mite antigen as in Example 2 were used as antigens. An antigen reagent was prepared by diluting with CTL-Test medium (C.T.L.) to an appropriate concentration.

### 2. Preparation and Measurement of Measurement Sample

A measurement sample to which each antigen reagent was added was prepared in the same manner as in Example 2. For comparison, a measurement sample to which the antigen reagent was not added was also prepared. For each measurement sample, fluorescence staining of pERK and CD4 was performed in the same manner as in Example 2. Each measurement sample after staining was measured with MI-1000 in the same manner as in Example 2.

### 3. Data Analysis

### Detection by Algorithm Analysis of Measurement Data

Algorithm analysis of measurement data was performed in the same manner as in Example 10, and the number of cell complexes containing an activated T cell per PBMC 1 × 10⁴ cells in each measurement sample was acquired. The number of cell complexes for the measurement sample not containing an antigen reagent was subtracted from the number of cell complexes for each measurement sample to which an antigen reagent was added. The obtained value was acquired as the number of cell complexes per PBMC 1 × 10⁴ cells in each measurement sample to which the antigen reagent was added.

### Detection by Visual Confirmation of Image

Data analysis was performed with IDEAS software in the same manner as in Example 2. The extracted image of the cell complex containing an activated T cell was visually confirmed, and the number of cell complexes of a T cell fluorescently stained with pERK and a B cell was counted in the same manner as in Example 10. The acquired number of cell complexes was converted into the number per PBMC 1 × 10⁴ cells to acquire the number of cell complexes containing an activated T cell per PBMC 1 × 10⁴ cells in each measurement sample. The number of cell complexes per PBMC 1 × 10⁴ cells in the measurement sample to which the antigen reagent was added was acquired in the same manner as in the above (1).

### 4. Results

FIG. 38 shows a graph obtained by plotting the number of cell complexes obtained by the algorithm analysis of measurement data of the above (1) and the number of cell complexes obtained by visual confirmation of the above (2). As shown in FIG. 38, with respect to the number of cell complexes containing an activated T cell per PBMC 1 × 10⁴ cells in each measurement sample, it was found that the detection result by the algorithm analysis of measurement data and the detection result by visual confirmation of an image were clearly correlated. Therefore, it was shown that by the analysis of measurement data by an algorithm, the cell complex containing an activated T cell can be detected with approximately the same accuracy as the visual confirmation of an image.

What is claimed is:

1. A method for detecting an antigen-specific activated T cell comprising:
acquiring first information on a particle size and second information on a T cell activation marker for a first measurement sample prepared by mixing in vitro a first specimen separated from a biological sample containing a T cell and an antigen-presenting cell and an antigen reagent containing a predetermined antigen, by measuring the first measurement sample with a flow cytometer;
acquiring first information on a particle size and second information on a T cell activation marker for a second measurement sample prepared from a second specimen separated from the biological sample and not containing the antigen reagent, by measuring the second measurement sample with the flow cytometer;
detecting a target particle in the first measurement sample based on the first information and the second information on the first measurement sample, and detecting a background particle in the second measurement sample based on the first information and the second information on the second measurement sample; and
detecting a cell complex in which the T cell and the antigen-presenting cell adhere to each other in the first measurement sample, the cell complex including a T cell activated by the predetermined antigen, based on a detection result of the target particle and a detection result of the background particle.

2. The method according to claim 1, wherein the biological sample is a blood specimen collected from a subject.

3. The method according to claim 1, wherein the predetermined antigen is a protein contained in a pathogen, a fragment of the protein, an allergen, a drug, an autoantigen, or a cancer antigen.

4. The method according to claim 3, wherein the pathogen is a bacterium or a virus.

5. The method according to claim 1, comprising preparing the first measurement sample and the second measurement sample before the acquiring the first information and the second information.

6. The method according to claim 1, wherein the first information is optical information reflecting the particle size.

7. The method according to claim 6, wherein the optical information is forward scattered light information of the particle.

8. The method according to claim 1, wherein the flow cytometer is an imaging flow cytometer, and the first information is an area value of the particle in an image captured by the imaging flow cytometer.

9. The method according to claim 1, wherein the second information is at least one selected from the group consisting of information on activation of mitochondria of the T cell and information on an amount of a predetermined molecule or a predetermined substance in the T cell.

10. The method according to claim 9, further comprising, before acquiring the first information and the second information, labeling at least one selected from the group consisting of the mitochondria, predetermined molecule, and predetermined substance in the T cell with a capture body capable of generating an optical signal.

11. The method according to claim 10, wherein the predetermined molecule is at least one selected from the group consisting of phosphorylated ERK, NF-κB, NFAT, IFNy and IL-2, and the predetermined substance is at least one selected from the group consisting of calcium ions and reactive oxygen species.

12. The method according to claim 1, wherein the flow cytometer is an imaging flow cytometer, and the second information is a value based on an optical signal, obtained from an image captured by the imaging flow cytometer.

13. The method according to claim 1, wherein in the detecting the target particle and the background particle,
a particle in which the first information on the first measurement sample is greater than or equal to a first threshold value corresponding to the first information and the second information on the first measurement sample is greater than or equal to a second threshold value corresponding to the second information is detected as the target particle, and
a particle in which the first information on the second measurement sample is greater than or equal to the first threshold value and the second information on the second measurement sample is greater than or equal to the second threshold value is detected as the background particle.

14. The method according to claim 1, wherein in detecting the cell complex, a value obtained by subtracting the number of background particles in the second measurement sample from the number of target particles in the first measurement sample is acquired as the number of cell complexes in the first measurement sample.

15. The method according to claim 14, wherein when the number of cell complexes in the first measurement sample is greater than or equal to a predetermined threshold value, it is suggested that the biological sample has been collected from a subject having a specific immune response to the predetermined antigen.

16. The method according to claim 1, comprising, in acquiring the first information and the second information, acquiring the first information on a particle size and the second information on a T cell activation marker antigen-specific activity for a third measurement sample prepared by mixing in vitro a third specimen separated from the biological sample, the antigen reagent, and an immune checkpoint inhibitor by measuring the third measurement sample with the flow cytometer;
in detecting the target particle and the background particle, detecting the target particle in the third measurement sample based on the first information and the second information on the third measurement sample; and
in detecting the cell complex in the first measurement sample, detecting the cell complex in the third measurement sample based on a detection result of the target particle in the third measurement sample and a detection result of the background particle in the second measurement sample.

17. The method according to claim 16, wherein in detecting the target particle and the background particle, a particle in which the first information on the third measurement sample is greater than or equal to the first threshold value and the second information on the third measurement sample is greater than or equal to the second threshold value is detected as a target particle in the third measurement sample.

18. The method according to claim 16, wherein in detecting the cell complex, a value obtained by subtracting the number of background particles in the second measurement sample from the number of target particles in the third measurement sample is acquired as the number of cell complexes in the third measurement sample, and
when the number of cell complexes in the first measurement sample is lower than the number of cell complexes in the third measurement sample, it is suggested that the biological sample has been collected from a subject who is in a state of immune tolerance to the predetermined antigen.
